(19) United States
(12) Patent Application Publication
(10) Pub. No.: US 2025/0049837 A1
(43) Pub. Date: Feb. 13, 2025

(54) MICRORNA-BASED PARTICLE FOR THE TREATMENT OF DYSREGULATED IMMUNE RESPONSE

(71) Applicants: UNITY HEALTH TORONTO, TORONTO (CA); NORTHMIRS, INC., Toronto (CA)

(72) Inventors: CLAUDIA CHIMISSO DOS SANTOS, TORONTO (CA); SAMANTHA MARIE MCWHIRTER, NEW RICHMOND (CA); AMIN MOTAMED EKTESABI, TORONTO (CA); JIM TSOPORIS, TORONTO (CA); CHIRAG MANOJ VASWANI, TORONTO (CA); GILBERT CREIGHTON WALKER, TORONTO (CA); LOGAN WALTER CHARLES ZETTLE, BRAMPTON (CA)

(21) Appl. No.: 18/933,178

(22) Filed: Oct. 31, 2024

Related U.S. Application Data
(63) Continuation-in-part of application No. 18/794,415, filed on Aug. 5, 2024.
Application No. 18/794,415 is a continuation of application No. 18/408,093, filed on Jan. 9, 2024.
(60) Provisional application No. 63/437,855, filed on Jan. 9, 2023.

(51) Int. Cl. A61K 31/712; A61K 9/00; A61K 9/51; A61K 31/7115; A61K 31/7125; A61P 9/10

(52) U.S. Cl. CPC A61K 31/712; A61K 9/0019; A61K 9/5123; A61K 9/5146; A61K 31/7115; A61K 31/7125; A61P 9/10

(57) ABSTRACT

A method for treating or preventing sepsis in a subject in need is disclosed. The method includes administering miRNA-based particles to a subject in need, and observing extended survival of a subject suffering from sepsis and reduced organ dysfunction or failure caused by the condition. Each of the miRNA-based particles includes miR-193b-5p inhibitor, encapsulated in a lipid nanoparticle carrier. The method is for treating dysregulated immune response induced by the sepsis. The method can be for treating acute respiratory distress syndrome, preventing or treating neuroinflammation, preventing lung injury, and any combination thereof. The lipid nanoparticle carrier includes an ionizable cationic lipid positively charged in a formulation buffer, a sterol, a structural helper lipid, and a PEGylated-lipid. Alternatively, the lipid carrier can be a cell membrane derived nano-vesicle.

### FIELD

The present disclosure relates to microRNA-inhibitor and microRNA mimic delivery for the treatment of dysregulated immune response including acute respiratory distress syndrome and myocardial dysfunction in sepsis.

### BACKGROUND

Acute Respiratory Distress Syndrome (ARDS) and acute lung injury (ALI, pre-clinical corollary) are disorders of acute inflammation that disrupt the lung endothelial and epithelial barriers.¹

Inciting events, such as microbial or viral infections or other injurious stimuli, may result in the dysregulated activation of the innate immune response leading to cell damage on either side of the alveolar-capillary membrane (epithelial and endothelial), loss of membrane permeability with an influx of edema fluid, and gap formation. Multiple therapeutics are important to consider in an overall strategy to protect against lung injury: modulating the cytokine storm, reducing edema, and repairing tight junction damage. The main barrier to therapeutic success may be inducing an unintended immune response.

A recent approach for treating lung injury has been toward mesenchymal stromal cell therapy and has unfortunate limitations due to delayed therapy. This shortcoming of MSC therapy underscores the need for off-the shelf, cell-free products that can be quickly and reliably deployed at the point-of-need. This indicates a benefit from using a synthetic carrier.

Recently, dos Santos and others have shown microRNAs (miRNAs) that are involved in the regulation of host responses to injury, inflammation and infection^{1,2,3,4,5,6}. MiRNAs are endogenous non-coding, small RNAs 18-24 nucleotides in length. microRNAs function via base-pairing with complementary sequences within messenger RNA (mRNA) molecules. miRNAs interfere with protein synthesis by directly degrading or inhibiting the translation of target genes. MiRNAs typically affect the expression of more than a single protein and influence multiple pathways. They differ, for example, from small interfering RNAs (siRNAs) that target specific proteins. microRNAs also differ from messenger RNA (mRNA) which are thousands of nucleotides in length and which provide a template for protein synthesis (protein translation). microRNAs' repressive function occurs inside the cell cytoplasm but outside the nucleus.

Nucleic acids are susceptible to degradation by endo- and exo-nucleases and so the delivery of nucleic acids in therapeutics has been accomplished by carriers such as virus shells, polymers and lipid nanoparticles. As examples, lipid nanoparticles (LNPs) were demonstrated in the first LNP gene therapy (Onpattro™) for liver amyloidosis and the COVID vaccines from Moderna and Pfizer. The LNPs comprise of several lipid types. A cationic ionizable lipid is used to condense anionic nucleic acid cargos. Other, structural lipids may, as non-limiting examples, create the shell of the carrier, increase its circulation time, stiffness, and help in the release of the cargo when desired. Specific targeting moieties can also be included on the LNP surface or conjugated to the nucleic acid.

Acute lung injury resulting in acute respiratory distress syndrome (ARDS) can be precipitated by a variety of causes including infectious and non-infectious triggers. The most common cause is a dysregulated response to infection (sepsis). Non-infectious causes, pancreatitis, aspiration of gastric contents, smoke inhalation, as well as inhalation of noxious gases, severe traumatic injuries with shock and blood product transfusion (i.e., transfusion-related acute lung injury) may also result in ARDS. More recently, new causes of ARDS have emerged including vaping-associated lung injury and drug-induced ARDS caused by a variety of agents such as immunotherapies including checkpoint inhibitors. Of relevance, is the increasing recognition of the role of viral infection as a significant cause of ARDS: SARS-CoV (2003), H1N1 influenza (2009), MERS-CoV (2012), and SARS-CoV-2 virus (2019) that led to the COVID-19 pandemic. Moreover, exposure to injurious mechanical ventilation may further exacerbate acute lung injury. Repetitive cyclic stretch, overdistension, alveolar collapse and deformation injury contribute significantly to increased lung injury and resulting ARDS related morbidity and mortality.

The pathophysiology of ARDS is incompletely understood. Diffuse alveolar damage is the pathognomonic histological finding associated with ARDS. This is characterized by neutrophilic alveolitis and hyaline membrane deposition. Damage to the alveolar capillary membrane is associated with denudation of the alveolar capillary membrane (cells detach from the membrane due to injury or death): this is associated with loss of membrane permeability, diffuse alveolar hemorrhage, proteinaceous exudative edema in alveolus, and hyaline membranes with fibrin rich deposits that form along the denuded alveolar basement membrane in areas of substantial epithelial and endothelial lung injury.

Damage to the tight lung epithelial barrier facilitates alveolar flooding and impairs the transport of fluid by the alveolar epithelium, the normal mechanism for maintaining a dry airspace. Injury to type II cells might impair surfactant production. Surfactant can also be inactivated by alveolar flooding. Concomitant injury and shedding of the lung epithelial glycocalyx, a layer of glycosaminoglycans and proteoglycans that covers the alveolar surface, is also proinflammatory. Activation and injury of the alveolar epithelium also leads to the shedding of anticoagulant molecules and the release of tissue factor from the lung epithelium into the alveolar space. These changes favour intra-alveolar fibrin formation, which drives hyaline membrane formation. The alveolar epithelium is an important barrier against pathogens and can secrete antibacterial proteins such as surfactant proteins A and D; therefore, epithelial injury can also increase susceptibility to secondary infection. The capillary endothelium forms the barrier between circulating blood cells and plasma and the lung interstitium and airspace. Injury to the lung endothelium is a key feature of ARDS and is characterised by the formation of gaps between endothelial cells and upregulation of adhesion molecules such as P-selectin and E-selectin and endothelial injury mediators such as angiopoietin-2. A variety of stimuli can trigger endothelial injury including circulating pathogens or their products, endogenous disease-associated molecular patterns, proinflammatory cytokines, and cell-free haemoglobin. Severe injury to the lung epithelium can also trigger injury to the lung endothelium. Although the mechanisms are not well understood, direct cell-to-cell communication and transfer of reactive oxygen species between lung epithelial and endothelial cells contributes to injury. As with the lung epithelium, the endothelium is covered with a glycocalyx that is easily injured and shed, exposing adhesion molecules and favouring oedema formation. Endothelial injury causes the shedding of anticoagulant molecules on the endothelial surface such as thrombomodulin and the endothelial protein C receptor, and upregulation of procoagulant molecules favouring microvascular thrombus formation.

Injury to the alveolar capillary membrane is responsible to the clinical features associated with ARDS—exudative pulmonary edema (flooding of the alveoli—associated radiographically with bilateral diffuse airspace disease), impaired gas exchange (presenting as severe hypoxemia), and increased work of breathing (presenting as respiratory distress). Alveolar flooding is further exacerbated by breakdown of normal fluid transport mechanisms in the lung—which normally pump alveolar edema into the interstitium to be cleared by the lymphatics. In addition to contributing the ventilation-perfusion mismatch, edema fluids contribute to the inactivation of surfactant leading to microatelectasis and end-expiratory alveolar collapse, and decreased lung compliance requiring higher inspiratory pressures and increased work for breathing. Activation of procoagulant pathways on the lung's endothelium can lead to lung microvascular thromboses which increase dead space (this is when ventilation occurs in non-perfused alveoli/areas of the lung); increased dead space ventilation contributes to severe gas-exchange impairments and is associated with higher mortality in ARDS. Unlike vessels in the systemic circulation, the response of the pulmonary circulation to hypoxia is to vasoconstrict—which leads to further hypoxemia and increased pulmonary vascular resistance. Microvascular thromboses and severe damage to the microvascular bed can further contribute to pulmonary arterial hypertension and acute right ventricular dysfunction, both of which contribute to poor clinical outcomes.

Local and systemic acute inflammation are prominent features of ARDS that contribute to lung epithelial and endothelial injury. Early in the course of ARDS—cell activation via pattern and damage recognition receptors leads to increased expression of adhesion molecules on epithelial and endothelial surface and secretion of cytokines and chemokines. Neutrophils are normally not found in the alveolar space. The increased secretion of neutrophil chemoattractants leads to the infiltration of neutrophils that are activated, and in the activated state can release a variety of injurious mediators including reactive oxygen species, proteases, and proinflammatory lipid-derived mediators such as prostaglandins and leukotrienes. Neutrophilic extracellular traps composed of DNA, histones, and proteases are also released into the airspace during these pathophysiological processes and can increase inflammation by activating the NRLP3 inflammasome which initiates local release of interleukin-1-β and interleukin-18. Neutrophil recruitment is mostly done by tissue resident and recruited macrophages (circulating monocytes that enter the lung and become activated macrophages); although interstitial fibroblasts may contribute. Macrophage pattern recognition receptors bind disease-associated or pathogen-associated molecular patterns, which activate macrophages to a proinflammatory phenotype leading to the release of proinflammatory cytokines and neutrophil chemoattractants such as interleukin-8. Lung epithelial and endothelial cells can also release neutrophil chemoattractants.

Exposure to repetitive cyclic stretch in this pro-inflammatory environment where cells are more susceptible to mechanical injury is associated with changes in mechanosensory, mechanotransduction, and pulmonary compartmentability (ultrastructural damage to the alveolar capillary membrane) leading to spillage of the “proinflammatory soup” into the circulation and ensuing distal organ damage—multiorgan dysfunction syndrome—including worsening acute kidney injury, acute encephalopathy, cardiomyopathy, hemodynamic instability, liver dysfunction, gut dysfunction and possibly muscle dysfunction contributing to increased morbidity, mortality and post-ARDS and post-ICU syndrome.

Resolution of inflammation is a coordinated process that requires downregulation of proinflammatory pathways and upregulation of anti-inflammatory pathways. T-regulatory cells have a vital role in coordinating this process. Neutrophils are cleared from the airspace through apoptosis and phagocytic clearance by alveolar macrophages. Proresolving mediators, including lipoxins and resolvins, are a family of bioactive lipid mediators that might also have a role in resolution of lung injury and inflammation. Restoration of alveolar epithelial fluid transport requires the regeneration of the alveolar epithelium, which can be necrotic in ARDS. Several cells can act as progenitors to repopulate the epithelium, and their relative roles might depend on the severity of epithelial injury. Once a tight epithelial barrier is restored, various endogenous factors can upregulate alveolar fluid clearance, including catecholamines and corticosteroids. The role of interstitial cells, such as fibroblasts, in the acute and resolution phases of ARDS is poorly understood. Profibrotic pathways are triggered as early as the first day of ARDS and can lead to lung fibrosis. Fibrosis can impede ventilator weaning, and cause long-term impairment of lung function with restrictive physiology and decreased diffusing capacity.

While no formal definitions exist for septic cardiomyopathy, the accepted diagnostic criteria includes acute, global biventricular reduced contractility, left ventricular dilation, diminished response to fluids and catecholamines, and absence of coronary syndrome^{7,8}. Presence of myocardial dysfunction is associated with 70-90% mortality, in contrast to 20% mortality in patients without myocardial involvement^{9,10}. Reduced left ventricular ejection (LV) fraction (LVEF<45%) occurs in 60% of patients with septic shock in the first 3 days⁷. In those patients who die, myocarditis is seen in 27% of autopsy samples, as well degenerative changes^{10,11}. The pathogenesis is complex and incompletely understood, involving a combination of immune dysregulation, oxidative stress, disorder of calcium regulation, autonomic nervous system dysregulation, endothelial dysfunction and catastrophic mitochondrial injury.^{11, 12,13} Myocardial dysfunction in sepsis is a treatable trait, and miRNAs may contribute to features of sepsis-induced cardiomyopathy.^{14, 15,16}

### SUMMARY

The present disclosure provides a method for treating or preventing sepsis in a subject in need, comprising steps of:
administering miRNA-based particles to a subject in need, and
observing extended survival of a subject suffering from sepsis and reduced organ dysfunction or failure caused by the condition;
wherein each of said miRNA-based particles comprises miR-193b-5p inhibitor, encapsulated in a lipid nanoparticle carrier.

In an embodiment, the step of administering the miRNA-based particles includes injecting a physiologically acceptable solution containing the miRNA-based particles into the subject.

In an embodiment, the step of administering the miRNA-based particles includes mixing the miRNA-based particles with microbubbles to adhere a plurality of the miRNA-based particles to outer surfaces of the microbubbles and injecting the microbubbles into a patient, followed by applying ultrasound at one or more targeted locations of the patient's body to rupture the microbubbles causing release of the miRNA-based particles, and/or the cargo of the miRNA-based particles.

In an embodiment, the method is for treating dysregulated immune response induced by the sepsis.

In an embodiment, the method is for treating acute respiratory distress syndrome induced by the sepsis.

In an embodiment, the method is for preventing or treating neuroinflammation induced by the sepsis.

In an embodiment, the method is for preventing lung injury.

In an embodiment, the miRNA-based particles further comprise miR-187-3p mimic encapsulated in a lipid nanoparticle carrier.

In an embodiment, the method is for any one or any combination of: treating acute respiratory distress syndrome, preventing or treating neuroinflammation, preventing lung injury, and treating or preventing myocardial dysfunction.

In an embodiment, the miR-193b-5p inhibitor is an miRNA sponge comprising synthetic or naturally occurring RNA molecules, said miRNA sponge having multiple binding sites for miR-193b-5p.

In an embodiment, the miR-193b-5p inhibitor is a synthetic single-stranded oligonucleotide having 18-24 monomers in length that bind to miRNA-binding region of target genes, wherein the miR-193b-5p inhibitor optionally further includes a longer guide strand integrated with one or more passenger strands.

In an embodiment, the miRNA-binding region is 3′-untranslated region of surfactant protein C gene.

In an embodiment, the miRNA-binding region is 3′-untranslated region of occludin gene.

In an embodiment, the lipid nanoparticle carrier comprises at least four (4) types of lipids, said at least four (4) types of lipids being independent from each other, and comprising:
a) an ionizable cationic lipid selected to be positively charged in a formulation buffer,
b) a sterol,
c) a structural helper lipid, and
d) a PEGylated-lipid.

The present disclosure provides miRNA-based particles, comprising:
any one or combination of miR-187-3p or miR-193b-5p inhibitor or mimics thereof; and
a lipid nanoparticle carrier comprising at least four (4) types of lipids, said at least four (4) types of lipids being independent from each other, and comprising: a) an ionizable cationic lipid selected to be positively charged in a formulation buffer, b) a sterol, c) a structural helper lipid, and d) a PEGylated-lipid.

The synthetic microRNA may be any one or combination of miR-193b-5pinh, miR-187-5p, hsa-miR-7107-5p, hsa-miR-6803-5p, hsa-miR-6798-5p, hsa-miR-760, hsa-miR-6727-5p, hsa-miR-4763-3p, hsa-miR-3652, hsa-miR-885-3p, hsa-miR-766-3p, hsa-miR-3175, hsa-miR-6893-5p, hsa-miR-6875-5p, hsa-miR-6799-5p and hsa-miR-6787-5p.

The ionizable cationic lipid may be positively charged in a formulation buffer having a pH in a range from about pH 3 to about pH 5.5.

The ionizable cationic lipid is neutral in a storage buffer having a pH in a range from about pH 7 to about pH 8. The storage buffer may be a phosphate buffered saline (PBS) having a pH of about 7.4.

The ionizable cationic lipid may be any one or a combination of saturated lipids, unsaturated lipids, single-tail lipids, multi-tail lipids, polymeric lipids, biodegradable lipids, or branched tail lipids.

The ionizable cationic lipid may comprise neutral or true fats, waxes, cutin, suberin, phospholipids, sphingolipids, lipoproteins, terpenes, prostaglandins, or sterols.

The sterol is selected to facilitate endocytosis via a low density lipid receptor by complexing with apolipoprotein E.

The sterol may be selected on the basis of improving intracellular delivery.

The structural helper lipid may be selected to contribute to lipid nanoparticle stability and/or enhances endosomal release.

The structural helper lipid may be a cylindrical-shaped lipid like phosphatidylcholine.

The structural helper lipid may be a cone-shaped geometry which favors the formation of the hexagonal II phase, and promotes endosomal release of oligonucleotides.

The structural helper lipid may be a sterol and the sterol may be cholesterol.

The PEGylated-lipid may be selected such that it stabilizes the particle and protects it from opsonization before reaching an intended target.

The PEGylated-lipid may be polyethylene glycol (PEG) derivatives attached to a lipid moiety.

The PEGylated-lipid may be DMG-PEG2000 or 1,2-dimyristoyl-rac-glycero-3-methoxypolyethylene glycol-2000 ALC-0159 or (2-hexyldecanoate), 2-[(polyethylene glycol)-2000]-N,N-ditetradecylacetamide, DSPE-PEG, DPPE-PEG, DOPE-PEG, DMPE-PEG with PEG lengths varying from 0.2 to 5 kDa.

The ratio of the ionizable lipid, the sterol, the structural helper lipid, and the PEGylated-lipid may be in a mol % ratio range of about 40-70:30-45:3-16:0.5-1.5.

[symbol] ratio of the ionizable lipid, the sterol, the structural helper lipid, and the PEGylated-lipid may be in a mol % ratio range of about 45-55:37-40:8-12:1-1.7.

The ratio of the ionizable lipid, the sterol, the structural helper lipid, and the PEGylated-lipid may be in a mol % ratio range of about 50:38.5:10:1.5.

The lipid carrier may further comprise any one or combination of a prodrug lipid, or a lipid carrier non-covalently functionalized by peptides, proteins, glycoproteins, polysaccharides or their combinations to aid tissue specific targeting. This prodrug lipid may include any one or combination of anti-inflammatories, inflammatories, disease-modifying anti-rheumatic drugs (DMARDs), chemokine receptor antagonists, immune response modifiers, immunomodulating drugs, Mast-cell stabilisers, T-cell activation inhibitors, TNF binding proteins, as non-limiting examples.

The composition of the at least four component lipid carrier may include this prodrug lipid in a mole fraction ranging from about 1 to about 15%, and is compensated for by removing the sterol and the structural helper lipid to compensate for the addition of the prodrug lipid, and wherein the fraction of prodrug lipid is in a range of about 5 to about 10%.

The present disclosure provides a composition comprising a microbubble and a plurality of the above-mentioned microRNA-based particles.

The plurality of the miRNA-based particles may be used to form a coating on an outer or an inner surface of the microbubble.

The composition may further comprise one or more prodrugs.

The present disclosure provides a medicament for treatment of dysregulated immune response, comprising the miRNA-based particles or the compositions disclosed above.

The dysregulated immune response may be acute respiratory distress syndrome.

The dysregulated immune response may be myocardial dysfunction in sepsis.

The dysregulated immune response may be any one or combination of acute kidney injury, acute encephalopathy, hemodynamic instability, liver dysfunction, gut dysfunction, and muscle dysfunction. All of these may contribute to increased morbidity, mortality and post-ARDS, post-cardiomyopathy and post-ICU syndrome.

The present disclosure provides a medicament for treatment of dysregulated immune response, comprising the miRNA-based particles disclosed above. The method of treatment comprises administering the miRNA-based particles to a subject.

Administering the miRNA-based particles includes mixing the miRNA-based particles with microbubbles to adhere a plurality of the miRNA-based particles to outer surfaces of the microbubbles and injecting the microbubbles into a patient, followed by applying ultrasound at one or more targeted locations of the patient's body to rupture the microbubbles causing release of the miRNA-based particles, and/or the cargo of the miRNA-based particles.

The dysregulated immune response may be acute respiratory distress syndrome.

The dysregulated immune response may be myocardial dysfunction in sepsis.

The dysregulated immune response may be any one or combination of acute kidney injury, acute encephalopathy, hemodynamic instability, liver dysfunction, gut dysfunction, and muscle dysfunction. All of these may contribute to increased morbidity, mortality and post-ARDS, post-cardiomyopathy and post-ICU syndrome.

The miR-187-3p or miR-193b-5p inhibitor is a synthetic single-stranded nucleic acid oligomer having 18-24 monomers in length designed to specifically bind to endogenous miRNAs that would otherwise bind to their target mRNA molecules and prevent their translation.

A backbone of the nucleic acids includes one or more phosphorothioate (PS) that replace the natural phosphor diester (PO) bond in naturally occurring nucleotides. The one or more ribose sugars of the nucleic acids may include modifications at the 2′-O positions, said modifications being 2′-O-methyl (2′-OMe), 2′-O-methoxyethyl (2′MOE), or 2′-fluoro (2′-F). The conformationally constrained analogues to RNA include constrained 2′-O-ethyl (cEt), locked nucleic acid (LNA), or 2′-O,4′-C-ethylene-bridged nucleic acids (ENA) prepared by putting in a methyl bridge from 2′-O to 4′-C positions of the ribose sugar, that replace one or more of the naturally occurring RNA bases.

The miR-187-3p or miR-193b-5p inhibitor includes peptide nucleic acids and phosphorodiamidate morpholino oligomers (PMOs) as charge-neutral nucleotides.

The miR-187-3p or miR-193b-5p inhibitor may be conjugated with lipid, peptide or sugar moieties at the 3′ end.

The miR-187-3p or miR-193b-5p inhibitor has a 3-carbon chain spacer (C3 spacer) at either the 3′ or 5′ end, or internally.

The miR-187-3p or miR-193b-5p inhibitor may have a 3′ or 5′ end phosphorylation.

The miR-187-3p or miR-193b-5p mimics are synthetic single- or multi-stranded nucleic acids having 18-24 monomers in length that bind to miRNA-binding regions of target genes. A backbone of the nucleic acids may include one or more phosphorothioate (PS) that replace the natural phosphodiester (PO) bond in naturally occurring nucleotides.

One or more ribose sugars of the nucleic acids may include modifications at the 2′-O positions, said modifications being 2′-O-methyl (2′-OMe), 2′-O-methoxyethyl (2′MOE), or 2′-fluoro (2′-F).

The nucleic acids may comprise conformationally constrained analogues to RNA. The conformationally constrained analogues to RNA includes constrained 2′-O-ethyl (cEt), locked nucleic acid (LNA), or 2′-O,4′-C-ethylene-bridged nucleic acids (ENA) prepared by putting in a methyl bridge from 2′-O to 4′-C positions of the ribose sugar, that replace one or more of the naturally occurring RNA bases.

The present disclosure provides an RNA collector consisting of synthetic or naturally occurring RNA molecules, wherein the RNA collector has multiple binding sites for target miRNAs, and traps the targeted miRNAs.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will now be described, by way of example only, with reference to the drawings, in which:

FIG. 1 is a diagram showing a lipid nanoparticle formulation as disclosed herein.

FIG. 2A shows RNA encapsulation efficiencies as determined by RiboGreen assay for example LNPs described herein.

FIG. 2B shows the hydrodynamic size (bars) and polydispersity indices (points) of the example LNPs described herein.

FIG. 2C shows the zeta potential of the example LNPs described herein.

FIGS. 2A, 2B and 2C therefore illustrate that standardized characterization methods confirm the quality of the LNPs. There are no significant differences between the LNPs by size, polydispersity, zeta potential.

FIG. 3 Mass spectrometry derived LNP compositions after preparation compared to lipid film. Cholesterol increases upon loading and decreases upon formation if no RNA is present. Ionizable lipid MC3 shows the opposite trend.

FIGS. 4A and 4B show two different LNP formulations (referred to as KC2 (FIG. 4B) and MC3 (FIG. 4A) based on its ionizable lipid), the addition of 2 mM CaCl₂ to the formulation buffer increased GFP mRNA translation to a significant degree at different levels of treatment.

FIGS. 5A, 5B, 5C and 5D show the effect of LNP mir-193b-5p INH of viral replication in vitro, in which:

FIG. 5A shows staining of OCln and E-cadherin along the perimeter of cells and overlay of tight and adherens junction proteins;

FIG. 5B shows 10 mM of siRNA FAM tagged delivered in BEAS2b cells for 24 hours. 100% transfection efficiency;

FIG. 5C shows miR-193b-p5 copies/μL contained in digital droplets relative to non-infected controls; and

FIG. 5D shows the expression of PR8 viral hemagglutinin (HA), RNA polymerase Subunit 1 (PB1), and neuraminidase (NA) was significantly reduced 24 hours post infection in cells treated with LNP miR-193b-5p INH compared to LNP with negative control (NC).

FIGS. 6A, 6B and 6C illustrate a non-limiting example of the biodistribution of LNP miR-193b-5p INH in PR8 infected mice in which:

FIG. 6A shows a schematic of experiment design. Mice were infected at time 0. Therapeutic or control empty particles were delivered at Day 4 post infection. Echocardiograms were performed on Day 6 after which mice were humanely sacrificed;

FIG. 6B shows fluorescence images of organs collected at Day 6. Images were taken at 6, 12, 24, 48, 72, 96, and 120 hrs intravenous injection. Ex vivo fluorescence images and corresponding optical intensity for major organs dissected at 24 h post-injection is shown;

FIG. 6C shows digital quantification of fluorescence in mock mice; and

FIG. 6D shows digital quantification of fluorescence in PR8 mice shows significant predominant recruitment of LNPs to spleen, liver, and lungs (N=3).

FIGS. 7A, 7B and 7C illustrate a non-limiting example of LNP-miR-193b-5pINH systemic administration at day 4 post H1N1/PR8-infection mitigates injury in WT mice with no overt harm to miR-193bKOs in which:

FIG. 7A shows lung Histology: Large square haematoxylin and eosin stain (40×) and immunohistochemistry for OCln stain (small square, bar=100 μm). LNP miR-193b-5p INH attenuates OCln loss in PR8 infected WTs. Arrows point to areas of OCln stain, distal bronchiole (Br.) alveolus (Alv.);

FIG. 7B shows bronchoalveolar lavage fluid (BALf) total cell and polymorphonuclear cell (PMN) count; and

FIG. 7C shows fold change (FC) in expression shows increased expression of miR-193b-5p target-gene OCln and decreased proinflammatory gene interleukin 6 (IL-6) relative to 36B4 (housekeeping gene) in mice that received the LNP-miR-193b-5pINH compared to scrambled. Circles represent values for individual mice (N=3-5).

FIG. 8A shows non-limiting predicted roles of miR-193b based on based on computationally predicted gene targets in Tarbase v8.0.

FIG. 8B further illustrates the nonlimiting predicted roles of miR-193b in the REACTOME.

FIG. 8C further illustrates the nonlimiting predicted roles of miR-193b in the KEGG pathway.

FIG. 8D further illustrates the nonlimiting predicted roles of miR-193b in the GO biological pathway.

FIG. 8E shows the non-limiting predicted role of miR-187-3p based on predicted and experimentally known gene targets in TarBase v8.0.

FIG. 8F shows the non-limiting predicted role of miR-187-3p based on experimentally verified gene targets in miRTarbase v8.0.

FIG. 9A shows exemplary data and a Schematic of MoA for miR-187-3p: Schematic of in vivo experiment.

FIG. 9B further shows mean±SEM of percent change in echo-derived Ejection Fraction.

FIG. 9C further shows TNFa in relation to 36-b4.

FIG. 9D shows probability of survival at 72 hrs post-CLP.

FIG. 9E shows results for neonatal cardiomyocytes that were exposed to LPS with ciMSC vs platelet EVs with or without Dynasore™ (DYN, inhibitor of endocytosis). Bars are mean±SEM of IL-6/18S.

FIG. 9F shows a Volcano plot to visualize 66 miRs found to be enriched in ciMSC-EVs compared to platelet EVs, including miR-187-3p and miR-574-3p (black squares).

FIG. 9G shows that miR-187-3p expression increased in hearts from septic mice that received ciMSC-EVs.

FIG. 9H shows box plots of miR-187-3p/U6 levels from formalin fixed paraffin embedded post-mortem human hearts from patients who died with sepsis versus non-septic.

FIG. 9I shows miR-187-3p copy number in whole blood from 9 patients enrolled in CISSI trial (Cellular Immunotherapy for Septic Shock), dosed with MSCs (0.3, 1, or 3 million cells/kg).

FIG. 9J shows a schematic of MoA for miR-187-3p. miR-187-3p is reduced during sepsis. miR-187-3p decreases the expression of TNFa, and we postulate IL-6 and S 100AI.

FIG. 10A shows delivery of LNP carrying miR-187-3p attenuates inflammatory gene expression, organ dysfunction and mortality. In vitro: A) Immunofluorescent image shows uptake of LNPs by primary murine neonatal cardiomyocytes treated with 10 nM of miR-187-3p mimic.

FIG. 10B further shows mean±SEM miR187-3p/U6 24 hrs after LNP-miR-187-3p delivery.

FIG. 10C further shows mean±SEM of TNFa/18s in response to LPS (mg/mL)±LNP-miR-187-3p for 24 hrs.

FIG. 10D further shows representative immunofluorescent images show LNP uptake in heart, lung, and spleen.

FIG. 10E further shows mean+SEM of percent left ventricular (LV) ejection fraction (EF) vs. CLP.

FIG. 10F further shows probability of survival at 48 hrs post-CLP in mice that received LNP-miR-187-3p compared to saline and empty LNP.

FIG. 10G further shows mean±SEM of TNFW18S in healthy tissue 48 hrs after CLP.

FIG. 10H shows a schematic of reconstitution experiment. Mice were randomized to receive ciMSC-EVs 6 hrs post CLP with either the miR-187-3p inhibitor or the miR-187-3p mimic.

FIG. 10I shows probability of survival, compared to CLP treated with saline.

FIG. 11A shows S100A1 is a target of miR-187-3p and S100A1 deficient mice are resistant to sepsis: Primary murine neonatal cardiomyocytes were transfected with the full-length S100A1 3′UTR fused to the luciferase (LUX) expression vector and treated with either miR-187 scrambled (SCR), miR-187-3p mimic (MIM) or inhibitor (INH); alone or with LPS for 24 hrs (A-B).

FIG. 11B further shows mean±SEM fold change (FC) in gene/18S

FIG. 11C further shows Western blot of S100A1 protein levels in cardiomyocytes.

FIG. 11D further shows results for S100A1KO-derived neonatal cardiomyocytes treated with LPS for 24 hrs. Bars are mean±SEM fold change (FC) in gene/18S, vs. control, and vs. LPS.

FIG. 11E shows mean±SEM relative to vehicle/empty vector showing increased expression of TNFα compared to LPS, vs. vehicle, vs. LPS

FIG. 11F shows probability of survival; vs. WT-CLP

FIG. 11G shows mean±SEM % ejection fraction (EF) measured at 48 hrs post-CLP, WT-Sham,

FIG. 11H shows plasma levels of S100A1 (ng/ml) measured at 24 and 48 hrs; mean±SEM, vs. pre-CLP.

FIG. 11I shows Western blot of changes in S100A1 protein levels in hearts from CLP-mice treated with LNP-miR-187-3p.

FIG. 12A shows novel miRs and targets: Schematic to test effects of the top 10 miRs identified in ci-EVs in human cardiomyocytes.

FIG. 12B results for neonatal cardiomyocytes transfected with an IL-6 3′UTR LUX expression plasmid or empty plasmid and treated with LPS. Mean+SEM of relative LUX activity vs. vehicle; vs. LPS.

FIG. 12C shows a box plot of miR-574-3p/U6 from formalin fixed paraffin embedded post-mortem human hearts from patients who died with sepsis.

FIG. 12D shows results for neonatal cardiomyocytes treated with LPS or miR574-3p mimic for 24 hrs. Bars are mean±SEM of miR574-3p/U6.

FIG. 12E shows further results for neonatal cardiomyocytes treated with LPS or miR574-3p mimic for 24 hrs, mean±SEM of S100A1/18S.

FIG. 12F shows further results for neonatal cardiomyocytes treated with LPS or miR574-3p mimic for 24 hrs, mean±SEM of IL-6/18S.

FIG. 12G shows mean±SEM of miR574-3p (copies/ng) in WT mice 48 hrs post CLP treated with saline or miRI87 LNP.

FIG. 13 shows enhanced cardiac delivery of miRNA using LNP combined with microbubbles and released using ultrasound.

FIG. 14 shows the benefit of the ultrasound-induced LNP release of miRNA as reflected in the improved left ventricle ejection fraction of rat heart.

FIG. 15A shows LNP-miR-193b-5p mimic downregulates Surfactant Protein C 3′UTR luciferase activity.

FIG. 15B shows LNP-miR-193b-5p mimic downregulates surfactant protein D 3′UTR luciferase activity.

FIG. 15C shows the effect of miR-193b-5p modulations on surfactant protein C (SPC) expression in BEAS2b cells exposed to viral (PR8) and bacterial (LPS) models.

FIG. 15D shows immunofluorescence demonstrating SPC protein (yellow) preservation in representative whole lung sections from miR-193b knockout (KO) mice relative to wild-type (WT) 6 days post-PR8/H1N1 infection.

FIG. 15E shows a representative Western Blot (WB) showing an attenuated decrease in SPC (Type C arrow) and SPD (Type D arrow) protein expression in miR-193b-5pKO compared to wild type over the 6 days post PR8/H1N1 infection.

FIG. 15F shows representative images of cytospin slides from the Bronchoalveolar lavage fluid (BALf) showing LNP-miR-193b-5p INH mitigates total cell infiltration in infected WT mice treated with LNP-miR193b-5p INH and protection in the miR-193b KO with or without treatment.

FIG. 15G shows WT mice have improved survival to 7 days post PR8/H1N1 infection when treated with the LNP-miR-193bp-5pINH versus the LNP-miR-SCR-INH.

FIG. 15H shows WT or miR-193bKO were randomized to mock or PR8/HIN1 infection treated at 4 days post-infection (dpi) intravenously with LNP-miR-193b-5pINH or LNP-miR-SCR-INH. Representative immunohistochemistry shows preservation of OCln in mice that received LNP-miR-193b-5pINH.

FIG. 15I shows representative Western blot showing improved lung SPC protein levels from PR8/H1N1 infected mice treated with LNP-miR-193b-5p INH or LNP-193b-5p mimic (MIM) in the WT and miR-193b KO mice.

FIG. 15J shows representative Western blot demonstrating relative preservation of lung SPC protein levels in infected mice treated with LNP-miR-193b-5p-INH compared to LNP-miR-193b-5p MIM. Bars are mean+SEM.

FIG. 15K shows Western blot densitometry showing preservation of lung SPC protein level expression from infected mice treated with LNP-193b-5p-INH. Bars are mean+SEM.

FIG. 15L shows Western blot densitometry showing loss of lung SPC protein level expression from infected mice treated with LNP-193b-5p-MIM. Bars are mean+SEM.

FIG. 16 shows flu-infected miR-193b KO mice show preservation of surface tension over 20 compression and expansion cycles to assess the surfactant biophysical functional activity from the Bronchoalveolar lavage fluid (BALf) collected 5 days post-PR8/H1N1 infection.

FIG. 17 shows lung tissue histology illustrating the beneficial effect of LNP-miR-187-3p after cecal ligation and puncture (CLP) two days after injection.

FIG. 18 shows representative gross images of mouse brains with an evident decrease in hemorrhage in miR-193b-5p inhibitor treatment through systemic injection relative to scrambled control 6 days post infection.

FIG. 19 shows occludin expression in the blood vessels of the hippocampus from animals infected with influenza and treated with LNP-miR193-5p-INH. Scale bars: 100 μm.

FIG. 20 shows improved preservation of occludin protein expression in the blood vessels in the hippocampus from PR8/H1N1 infected mice treated with LNP-miR193-5p-INH. Quantitative and comparative analysis of % positive stained both for occludin+IB4 and normalized to the non-infected animals brain tissues.

FIG. 21 shows astrocytes from mouse hippocampal regions using brain coronal sections show decreased reactivity to PR8/H1N1 infected mice treated with LNP-miR193-5p-INH and miR-193b KO mice, relative to scrambled or WT control, respectively. Scale bars: 100 μm and 50 μm.

FIG. 22 shows quantitative analysis using the mean intensity values (AU) to measure astrocyte reactivity in the hippocampus of infected animals treated with the LNP-miR193-5p-INH or LNP-miR-INH-SCR (n=3 to 5 per group).

FIG. 23 shows brain coronal sections showing astrocytes more intimately associated with blood vessels in the hippocampal brain region of infected animals treated with the LNP-miR193-5p-INH relative to the LNP-miR-INH-SCR. WT (top row) and KO (bottom row) mice were counterstained with DAPI, Isolectin B-4 (IB4-blood-vessels), and immunolabeled for GFAP. Scale bars: 100 μm.

### DETAILED DESCRIPTION

Various embodiments and aspects of the disclosure will be described with reference to details discussed below. The following description and drawings are illustrative of the disclosure and are not to be construed as limiting the disclosure. The drawings are not necessarily to scale. Numerous specific details are described to provide a thorough understanding of various embodiments of the present disclosure. However, in certain instances, well-known or conventional details are not described in order to provide a concise discussion of embodiments of the present disclosure.

### Definitions

As used herein, the terms, “comprises” and “comprising” are to be construed as being inclusive and open ended, and not exclusive. Specifically, when used in this specification including claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. Unless otherwise specified, the terms “about” and “approximately” mean plus or minus 25 percent or less. It is not the intention to exclude embodiments such as these from the present disclosure.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

As used herein, microRNA (miRNA) refers to endogenous non-coding, single-stranded small RNAs approximately 18-24 nucleotides in length. They may be longer, e.g., as much as 30 nucleotides in length. Functionally, miRNAs hinder protein synthesis by directly degrading or inhibiting the translation of target genes at the post-transcriptional level. There are two main types of miRNAs, intracellular miRNAs and circulating miRNAs, which are differentially expressed under different pathological and physiological conditions. As expression regulators, miRNAs target about 60% of protein coding genes, exerting a vital role in biological processes, such as but not limited to cell proliferation, apoptosis, differentiation, immunity, and inflammation. MiRNAs are typically characterized by their stability, specificity, and selectivity. Compared to traditional therapeutics that target specific receptors or proteins, the strategy of using miRNA has additional novelty since miRNAs typically affect the expression of more than a single protein and influence multiple pathways. At the target, miRNAs can generate strong, sustained, and comprehensive biological effects through endogenous gene silencing at nanomolar concentrations.

As used herein, the phrase “synthetic microRNA (miRNA)” or “miRNA mimic” refers to a synthesized mimic of naturally occurring miRNA. Synthetic miRNA has some or all of the functions of naturally occurring miRNA, and it may also have additional functions. For example, synthetic miRNA may have improved resistance to exo- and endo-nucleases compared with natural miRNAs. Synthetic microRNA in a delivery system may have enhanced endosomal release and intracellular targeting that could provide improvements to therapy compared to naturally occurring microRNA. Other advantages of synthetic microRNA compared with natural miRNA could be enhanced features such as improved loading into carriers, improved therapeutic efficacy, improved cell-specific targeting and improved safety. Non-limiting examples of synthetic microRNAs include, but are not limited to, ones that include the following features:
1.1 synthetic miRNA of 20-24 bp in length, where the miRNA is duplexed and has one or more passive passenger strands.
1.2 terminal modification: phosphorothioate to reduce nuclease activity; PS linkers at the 3′ and 5′ ends reducing nuclease activity;
1.3 produced using engineered polymerases with differing amounts of PS at the end to incorporate the following—and a C3 spacer;
1.3.1 2′-O-methyl bases (increases thermal stability; more resistant to endonucleases; have been delivered in vivo);
1.3.2 2′-F-purines (to increase Tm′);
1.3.3 N1-methyl-ψ (can only use its Watson-Crick face to base-pair with another nucleoside, thus preventing it from wobble-pairing with other nucleotides (G, U, and C) The RNA modifying enzyme, Nep1 can methylate the N1 position.
1.3.4 2′-O-methoxy-ethyl bases (2′MOE), 1.3.5 locked nucleic acids (LNA). 1.3.6 modifications including glycans and peptides or their combinations, e.g., at the mismatched or wobble nucleic acids.
   Herein synthetic miRNA and naturally occurring miRNA are both referred to as miRNA.

Another group of synthetic miRNAs is miRNA inhibitors, which can be used to suppress miRNA. A common approach to correct aberrant miRNA expression is based on the synthesis of antisense oligonucleotides with a complementary sequence.¹⁷ miRNA inhibitors are often synthetic single-stranded nucleic acids 18-22 monomers in length designed to specifically bind to endogenous miRNAs that would otherwise bind to their target mRNA molecules and prevent their translation. “Sponge” inhibitors may bind more than one copy of miRNA. miRNA inhibitors are commonly synthesized and they may have non-natural nucleic acid moieties to enhance their function. Non limiting examples include sugar modifications—20-OMe: 20-O-methyl, 20-F: 20-fluoro-RNA, LNA: locked nucleic acid, UNA: unlocked nucleic acid, and 20-MOE: 20-O-methoxyethyl; Backbone modifications—PO: phospho-diester, PS: phosphorothioate, PACE: phosphonoacetate, PMO: phosphorodiamidate morpholino oligomers, and PNA: peptide nucleic Acid. A miRNA inhibitor may not have a passenger strand. A miRNA inhibitor may be composed of multiple passenger and multiple guide strands or both together

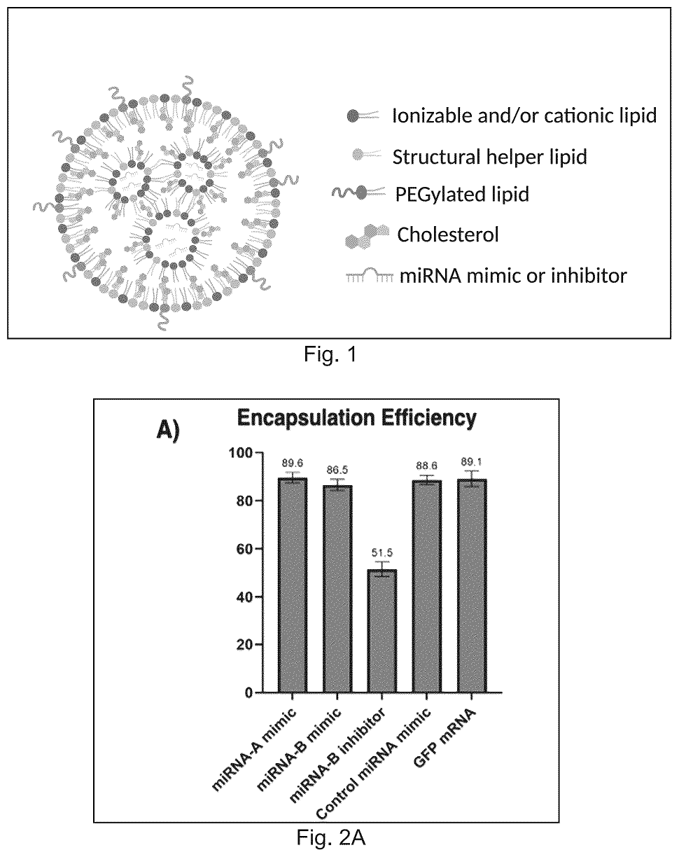

We provide several specific and non limiting examples of LNPs that can be prepared using microfluidics. As illustrated in FIG. 1, typical LNP formulations comprise four lipids: an ionizable lipid, cholesterol, a structural helper lipid, and a PEGylated-lipid. The ionizable cationic lipid is positively charged in the formulation buffer (n the range about pH 3.5-5.5, and preferably about pH 4), which binds and protects the negatively charged miRNA, and facilitates endosomal escape, but is neutral in the storage buffer (PBS, pH 7.4).¹⁸ Cholesterol is essential in the structure of the LNP, and is important for endocytosis via the LDL receptor by complexing, e.g., with apolipoprotein E or its variants. The PEGylated lipid stabilizes the particle and protects it from opsonization before reaching the intended target.

As used herein, the term “ionizable lipid” pertains to a lipid containing an ionizable group. This ionizable group can interact favorably with either microRNA or a microRNA inhibitor. In the event of a positive ionization, its role may involve binding to negatively charged microRNA, thereby facilitating the loading of the combination into a carrier. Should the carrier enter a cell's endosome, the ionizable lipid may possess a pKa that enables enhanced release from the carrier and endosome, adjusting to the changing pH within the endosome as it evolves over time or with maturation. This enhancement could result from alterations in the affinity of the ionizable lipid to the microRNA or changes in the interaction between the ionizable lipid and the carrier or endosome wall. This ionizable lipid may contain one or more ionizable groups. The pKa of the ionizable group(s) may be situated within the range of pKa=4 to pKa=6.9.

If the ionizable group is an amine, the lipid headgroup may feature a primary, secondary, or tertiary amine. The ionizable lipid may take the form of a branched molecule. It is imperative for the ionizable lipid to be amphiphilic, possessing an aliphatic or hydrophobic tail characterized by varying degrees of length, branching, or both. The aliphatic segment may incorporate carbons or sulfurs, and the bonds connecting them could be saturated or unsaturated, exhibiting different isomers. The lipid selection can be tailored to yield either a monolayer or a multilayer structure around the microRNA. It may be chosen to interact with additional lipids to enhance loading of the microRNA.

Non-limiting examples of structural classes of ionizable lipids encompass unsaturated lipids (containing unsaturated bonds), multi-tail lipids (with two or more tails), polymeric lipids (containing polymers or dendrimers), biodegradable lipids (featuring biodegradable bonds), and branched tail lipids. Combinations of these structural features within a specific ionizable lipid are possible. As used herein, the term “lipids” refers to neutral or true fats, waxes, cutin, suberin, phospholipids, sphingolipids, lipoproteins, terpenes, prostaglandins, and sterols.

As used herein, the term “lipids” encompasses neutral or true fats, waxes, cutin, suberin, phospholipids, sphingolipids, lipoproteins, terpenes, prostaglandins, and sterols.

Additionally, the term “phospholipid” is defined as a lipid molecule comprising a hydrophilic “head,” consisting of one or more phosphate groups, and two hydrophobic fatty acid “tails.” Typically, these components are connected by a glycerol molecule, making the phospholipid preferably a glycerol-phospholipid. Moreover, the phosphate group is often modified with simple organic molecules such as choline (yielding phosphocholine) or ethanolamine (resulting in a phosphoethanolamine).

Example phospholipids relevant to the invention can be selected from the list comprising but not limited to: 1,2-Dioleoyl-sn-glycero-3-phosphoethanolamine (DOPE), 1,2-Dioleoyl-sn-glycero-3-phosphocholine (DOPC), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-0-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

Other non-limiting examples of phospholipids include:

### Phospholipids:

| Acyl chains* | Headgroups** |
|---|---|
| 6:0/6:0 | PC, PE, PG, PS |
| 7:0/7:0 | PC, PE |
| 8:0/8:0 | PC, PE, PG, PS |
| 10:0/10:0 | PC, PE, PG, PS |
| 11:0/11:0 | PC, PE, PG, PS |
| 12:0/12:0 | PC, PE, PG, PS |
| 13:0/13:0 | PC, PG |
| 14:1c9/14:1c9 | PC |
| 14:0/16:0 | PC |
| 14:0/14:0 | PC, PE, PG, PS |
| 14:0/18:0 | PC |
| 15:0/15:0 | PC, PE |
| 16:0/2:0 | PC |
| 16:0/14:0 | PC, PG, PS |
| 16:0/16:0 | PC, PE, PG, PS |
| 16:1c9/16:1c9 | PC, PE |
| 16:4me3, 7, 11, 15/16:4me3, 7, 11, 15 | PC, PE, PG |
| 16:0/18:0 | PC |
| 16:0/18:2c9, 12 | PE |
| 16:0/18:1c9 | PC, PE, PG, PS |
| 16:0/20:4c5, 8, 11, 14 | PE |
| 17:0/17:0 | PC, PE, PG |
| 18:0/14:0 | PC, PE |
| 18:0/16:0 | PC, PG |
| 18:0/18:0 | PC, PE, PG, PS |
| 18:0/18:1c9 | PC, PE, PG, PS |
| 18:0/18:2c9, 12 | PE, PG |
| 18:0/22:6c4, 7, 10, 13, 16, 19 | PE |
| 18:1c9/14:0 | PC, PE |
| 18:1c9/18:1c9 | PC, PE, PG, PS |
| 18:1c9/16:0 | PC, PG |
| 18:1c9/18:0 | PC |
| 18:1t9/18:1t9 | PC, PE, PG, PS |
| 18:1y17/18:1y17 | PS |
| 18:2c9, 12/18:2c9, 12 | PC, PE, PG, PS |
| 18:2c9, 12/16:0 | PG, PS |
| 18:3c9, 12, 15/18:3c9, 12, 15 | PC, PE, PG |
| 19:0/19:0 | PC |
| 20:4c5, 8, 11, 14/16:0 | PG, PS |
| 20:0/20:0 | PC, PE, PG, PS |
| 20:4c5, 8, 11, 14/18:0 | PG |
| 20:4c5, 8, 11, 14/20:4c5, 8, 11, 14 | PE, PG |
| 21:0/21:0 | PC |
| 22:1c13/22:1c13 | PE, PG, PS |
| 22:0/22:0 | PC |
| 24:0/24:0 | PC |
| 22:6c4, 7, 10, 13, 16, 19/16:0 | PE, PG, PS |
| 22:6c4, 7, 10, 13, 16, 19/18:0 | PG, PS |
| 22:6c4, 7, 10, 13, 16, 19/22:6c4, 7, 10, 13, 16, 19 | PC, PE, PG, PS |

*The notation “Length of carbon chain:modifications” is employed, where modifications are represented by “0” for the default saturated configuration or the specific number of modifications. “C” and “t” denote cis and trans configurations of double bonds, “y” signifies a triple bond, and “me” indicates methyl isobranching, all followed by the carbon atom position of the modification.
**Headgroups refer to phosphatidylcholine (PC), phosphatidylethanolamine (PE), phosphatidylglycerol (PG), and phosphatidylserine (PS).

As used herein, “helper lipids” are lipids that contribute to the stability of lipid nanoparticles or enhance their delivery. An example of a helper lipid providing greater stability is a cylindrical-shaped lipid like phosphatidylcholine. Conversely, a helper lipid with a cone-shaped geometry may favor the formation of the hexagonal II phase, promoting endosomal release of oligonucleotides. Dioleoylphosphatidylethanolamine (DOPE) is an example of a helper lipid promoting endosomal release.

Another helper lipid promoting endosomal release is a sterol, such as cholesterol. Cholesterols can be classified into three regions: head, body, and tail. Examples include vitamin D3, vitamin D2, calcipotriol (Group 1); stigmasterol, beta-sitosterol (Group 2); and betulin, lupeol, ursolic acid, oleanolic acid (Group 3). A preferred embodiment is beta-sitosterol.

The term “PEGylated-lipid,” or PEG lipids, refers to polyethylene glycol (PEG) derivatives attached to a lipid moiety (e.g., DMG or DSPE). PEG lipids serve various functions, including increasing circulation time, resistance to aggregation, and reducing non-specific uptake for liposome-encapsulated (LNP) drugs. PEG lipids impact lipid nanoparticle properties by influencing particle size, stability, blood circulation time, and targeted delivery. The extent of these effects depends on PEG lipid proportions and properties, such as PEG molar mass and lipid length. Differences in circulation times among lipid nanoparticle-miRNA formulations may be attributed to the faster dissociation of a particular PEG lipid, enhancing cellular uptake and endosomal escape.

Non limiting examples of PEG-lipids include: DMG-PEG2000 or 1,2-dimyristoyl-rac-glycero-3-methoxypolyethylene glycol-2000 ALC-0159 or (2-hexyldecanoate), 2-[(polyethylene glycol)-2000]-N,N-ditetradecylacetamide, DSPE-PEG, DPPE-PEG, DOPE-PEG, DMPE-PEG (with PEG lengths varying from 0.2 to 5 kDa). Further non-limiting examples of PEG-lipids is given in Table 1.

TABLE 1
Table of PEG-lipids (adapted from ref 18))

| | | | PEG or |
|---|---|---|---|
| Common name | | | methoxy- |
| (if applicable) | Headgroup | Acyl chains* | PEG (mPEG) |
| DSPE-PEG | PE | 18:0/18:0 | PEG |
| DPPE-PEG | PE | 16:0/16:0 | PEG |
| DOPE-PEG | PE | 18:1c9/18:1c9 | PEG |
| DMPE-PEG | PE | 14:0/14:0 | PEG |
| | PE | 18:1c9/16:0 | PEG |
| | PE | 12:0/12:0 | PEG |
| | PE | 18:2c9, 12/18:2c9, 12 | PEG |
| DMG-PEG | Glyceride | 14:0/14:0 | mPEG |
| DSG-PEG | Glyceride | 18:0/18:0 | mPEG |
| DPG-PEG | Glyceride | 16:0/16:0 | mPEG |
| | Glyceride | 18:1c9/18:1c9 | mPEG |
| DSPE-mPEG | PE | 18:0/18:0 | mPEG |
| DPPE-mPEG | PE | 16:0/16:0 | mPEG |
| DMPE-mPEG | PE | 14:0/14:0 | mPEG |
| | PE | 18:1c9/18:1c9 | mPEG |
| ALC-0159 | Amino group | 14:0/14:0 | mPEG |
| | Amino group | 12:0/12:0 | mPEG |
| | Amino group | 12:0/14:0 | mPEG |
| | Amino group | 16:0/16:0 | mPEG |
| | Amino group | 18:0/18:0 | mPEG |
| PEG-cholesterol | Cholesterol | None | PEG |
| mPEG-cholesterol | Cholesterol | None | mPEG |

The notation employed is “Length of carbon chain:modifications,” where modifications are represented by “0” for the default saturated configuration or the specific number of modifications. “C” and “t” denote cis and trans configurations of the double bond, followed by the carbon atom position of the modification.

### Pro-Drug Lipid

The term “prodrug lipid” refers to lipidic prodrugs or drug-lipid conjugates wherein the drug is covalently bound to a lipid moiety, such as a diglyceride, a phosphoglyceride, or a fatty acid. The rationale for using a prodrug with a lipid nanoparticle (LNP) is based on the notion that the delivery of nucleic acids may necessitate vehicles like LNPs, which can potentially intensify immune stimulation. To mitigate this, potent immunosuppressive agents like dexamethasone have been co-administered with LNP formulations of siRNA. For example, an anti-inflammatory prodrug may effectively suppress the production of cytokines following intravenous administration of immune stimulatory oligodeoxynucleotides loaded in LNPs. LNPs tend to accumulate in phagocytic cells of the immune system after systemic administration, and the direct association of a prodrug with LNP enables more specific delivery to immune cells, potentially reducing the required dose.

Incorporating low levels of prodrug in LNPs containing unmodified mRNA, miRNA, or plasmid DNA, as compared with higher does of free drug, may significantly reduce pro-inflammatory cytokine levels following intravenous administration.²⁰ This may be achieved, in a non-limiting example, by chemically conjugating lipophilic acyl/alkyl moieties to drug or prodrug via biodegradable linkers, allowing its incorporation into the nanoparticle.

A simple test may illustrate the advantage of incorporating the prodrug into the lipid nanoparticle compared to injecting the drug separately. For instance, injecting LNPs containing modified miRNA in the interscapular space of the skin may result in skin lesions due to an immune reaction. Administering drug before LNP-miRNA injection may ameliorate this response, showcasing the potential benefits of incorporating the prodrug. The efficacy of miRNA in the presence of the prodrug lipid may be demonstrated to ensure its effectiveness is maintained.

This could be compared with any improvements observed when mice are injected with LNP-miRNA containing (e.g., 10 mol % of prodrug; fractions of lipids other than the ionizable lipids may be preferably reduced to maintain nucleic acid loading).

It may be advantageous to show that the prodrug lipid (e.g., of 10 mol %) does not reduce the efficacy of miRNA compared to the case of LNPs that do not carry pro-drug lipids. The immunomodulatory advantage of adding the pro-drug lipid may not be limited to LNPs with nucleic acid cargoes, it may also be advantageous for integration other species co-carried with the LNP, for example proteins or complex sugars.

Other components that may be advantageous to include in the lipid nanoparticle include but are not limited to the following: proteins, peptides, glycans, small molecule (<300 Dalton) drugs that may be hydrophobic, hydrophilic or of intermediate hydrophobicity, aggregates of these, whether preformed or not, metal nanoparticles, molecules that may serve to offer triggerable release of the nucleic acid, drug or prodrug cargo, or other functions such as enhancement of lipid nanoparticle uptake by cells, enhanced release of agent from LNPs once within cells, or enhanced targeting to different tissues, cell types, organelles with cells or macromolecular complexes within the cell cytoplasm or nucleus, as non-limiting examples.

Other advantage additions to the lipid nanoparticle may include increased cholesterol fraction to enhance endosomal release, divalent ions, varied N/P ratio, varied anionic/cationic lipid ratio optimize tissue targeting, alternative ionizable lipids, varied alkyl chains including variations such as chain length, position and isomer of double bonds, as well as S substitutions for C. These may be characterized for example, by diameter, zeta potential, lipid composition using LC-MSMS, loading, and transfection efficiency (GFP mRNA), unloaded as well as loaded.

### How to Make Nanoparticles

A non-limiting and summarizing description of how to make the lipid nanoparticles is as follows: DLin-MC3-DMA, DSPC, Cholesterol, and DMG-PEG2000 are combined at a 50:10:38.5:1.5 molar ratio in ethanol, miRNA is suspended in acetate buffer. The miRNA and lipid solutions are then mixed with a herringbone microfluidic mixer to form the LNPs. The resulting mixture is then purified via centrifugal filtration before storage in PBS.

A more detailed and still non-limiting description for making the LNP by including a lipid film in the preparation steps is as follows. A lipid film containing DLin-MC3-DMA, DSPC, Cholesterol, and DMG-PEG2000 at a 50:10:38.5:1.5 molar ratio may be prepared by combining the separate lipid components dissolved in chloroform. For LNP formulation, the aliquots may then dried with inert gas (N₂ or Ar) then placed in a vacuum chamber overnight to remove residual solvent. Films may then stored at −20° C. On the day of formulation, the lipid film may be dissolved in ethanol at a concentration of 6.8 mM (3.6 mg total lipid in 1 mL ethanol). The miRNA solution may be prepared by suspending 5 nmol miRNA mimic or inhibitor in 1200 uL 0.1 M acetate buffer (pH 4). The miRNA and lipid solutions may then mixed via microfluidic mixing with a flow rate ratio of 3:1 respectively, and a total flow rate of 9 mL/min. After mixing, the LNPs may be diluted to 12 mL with PBS (pH 7.4) and may be purified by filtration with a 30 kDa cut-off centrifugal filter before resuspension in PBS, then sterile-filtered through a 0.45 micron membrane syringe filter. For characterization, a 50 μL aliquot may be collected and diluted 20 fold in PBS and size, polydispersity index (PDI) and zeta-potential may then measured. The concentration and median size of the LNPs may determined, e.g., by nanoparticle tracking analysis at a dilution factor of, e.g., 2×10⁵ particles per milliliters (ml) The LNPs may then stored in PBS at 4° C. prior to use.

Following filtration of LNPs, encapsulation efficiency may be measured. Loading of LNPs may be quantified using a fluorescence based assay. To ensure accurate quantification, a standard curve may be created utilizing the same RNA which was loaded into the LNPs. Encapsulation efficiency may be calculated using the following formula:

$$E E = ( \frac{encapsulated miRNA}{total miRNA} ) \times \times 100 \%$$

### Nanovesicles as Alternative miRNA/miRNA-INH Carriers

Another class of carrier of nucleic acid therapeutics (siRNA, mRNA, miRNA, miRNA inhibitor, etc) is cell-membrane derived nano-vesicles.²¹ These nano-vesicles have numerous beneficial features, they exhibit long circulation lifetimes, and high biocompatibility, due to their composition being that of the cellular membrane. Additionally, as these vesicles contain approximately the same composition of the cell type they are derived from (in terms of lipids, proteins, glycans, etc) they are capable of endogenously targeting similar tissues as that of the cell line of which the nano-vesicle is derived. It is conceivable to create these nano-vesicles from isolated human cells of the following types, or cell lines and all possible sub lineages as listed below.

Bone Marrow Stromal Cells, non-limiting examples of which may include HS-5, HS-27A Myeloblasts, non-limiting examples of which may include HL-60, CMK, HEL, K-562, KASUMI-1, KG-1, LAMA-84, M-07e, MONO-MAC-1, MV4-11, NB-4, OCI-AML2, OCI-AML5, SIG-M5, THP-1, AR230, KCL22, U-937, Isolated Primary Human Neutrophils; Eosinophils, non-limiting examples of which may include EoL-1, AML14, AML14.3D10; Mast Cells, non-limiting examples of which may include HMC-1, LAD-1, LAD-2, LUVA Lymphoblasts, non-limiting examples of which may include SR4;11, MOLT-4, CCRF-CEM, BDCM; lung epithelial, non-limiting examples of which may include Primary Small Airway Epithelial Cells; Normal, Human (HSAEC), Beas2B, A549; and endothelial cell lines, non-limiting examples of which may include 293; Primary Umbilical Vein Endothelial cells; Normal, Human (HUVEC), and Primary Aortic Endothelial cells; normal, human (HAEC).

These nano-vesicles have an added benefit of being loadable, as hydrophilic cargo, such as small molecules, proteins, nucleic acids, etc. can be loaded into these nano-vesicles either passively during the cavitation process, or actively through pH gradient loading following cavitation and isolation of nano-vesicles. Hydrophobic drugs may be incorporated into the nano-vesicles either during the cavitation process, or by addition into solution with the purified nano-vesicles, where they spontaneously incorporate with the lipid bilayer.

Non-limiting examples of potential cargo to be loaded into the nano-vesicles are small molecules such as Cisplatin, TPCA-1, Piceatannol, Resolvin D2, Ceftazidime and Resolvin D1; and nucleic Acids such as DNA Plasmids, siRNA, mRNA, miRNA, miRNA inhibitors.

### Microbubbles as miRNA Delivery Agents When Combined With LNPs Carrying miRNA

Microbubbles are typically 0.5-10 μm in size. The gas inside a microbubble is typically a perfluorinated gas: e.g., perfluoropropane (C₃F₈), perfluorobutane (C₄F₁₀), and sulfur hexafluoride (SF₆) have been approved for biomedical use. The gas inside in a microbubble is surrounded or contained by a membrane that may consist of polymers, biocompatible biopolymers, surfactants, proteins, lipids, or a combination of these. The membrane may be a molecular fluid with its molecular components mixing to differing degrees, or it may be made from a membrane composed of nanoparticles packed at the surface. Different methods of preparation of microbubbles include sonication, cross-linking polymerization, atomization, reconstitution, and evaporation of solvent emulsion. Ultrasound is often preferred for preparation of microbubbles, e.g., by an ultrasonic needle in a syringe contacting a gas and a surfactant. The resulting microbubbles in the syringe may then be injected in a patient.

One use for microbubbles is to apply them as a drug carrier, where the drug can be additionally loaded in the microbubble in the syringe, later to be released in the patient by additional, applied ultrasound. Microbubbles therefore can prevent the release of substances before they have reached an area of therapeutic and/or diagnostic interest.

Differing compositions of the microbubble shell, e.g., varying neutral, anionic phospholipids, and polyethylene glycol-conjugated phospholipids at appropriate ratios, can affect the relative stability of microbubbles for ultrasound-based imaging or drug delivery.

Nanoparticles carriers of substances can be combined with microbubbles for local delivery using ultrasound. The microbubbles and lipid nanoparticles may be combined before administration (e.g., by intravenous injection) or they may be administered at different times. Microbubbles excited by ultrasound can be used to transient permeability of tissue or tissue components through which nanoparticles can then pass, to enhance delivery of the contents of nanoparticles to desired locations, e.g., at the focus of the ultrasound.²²

### Formulation for Use for Making Coated Microbubbles

Depending on their composition, nanoparticles may fuse (mix fully or partially) with a surfactant stabilizing the microbubble or nanoparticles may independently act as surfactants to stabilize a microbubble. In the second case, the nanoparticle may have its composition altered by its function as a surfactant that stabilizes a microbubble or the nanoparticle composition may not be altered by its function as a surfactant stabilizing the microbubble. In this last case, the nanoparticle might be in some cases be viewed as a nanoparticle coating. The composition of the nanoparticle can be such to have individual components that interact separately with the gas inside and the liquid outside. Or the nanoparticle may have a surface energy that is intermediate and between that of the gas and the liquid, and hence stabilize microbubbles. A combination of these two properties may also be advantageous.

### Mirna-Mimic Identity Used for Making Microbubbles

In the case of a solid lipid nanoparticle containing microRNA to be used as a therapeutic in conjunction with a microbubble delivery approach, the lipid nanoparticle may retain its identity as an intact particle or its components may disburse within a membrane or to create a membrane. The miRNA-mimic composition may be expected to affect the extent to which the nanoparticle intactness or dispersal occurs. This will depend on the stability the lipid nanoparticle. The transfection event after ultrasound triggered local release of miRNA mimic may be enhanced if the lipid nanoparticle is largely intact, as certain sizes of miRNA-mimic aggregates traverse cell membranes preferentially.

The release profile of the miRNA-mimic maybe expected to depend on the stability of the miRNA-mimic in its environment and the strength of the externally applied ultrasound that would release it.

MicroRNA-inhibitors may also be administered as above with microbubbles.

### Utility

Non-limiting examples of utility are as follows. The nanoparticles can be used to deliver an agent that acts as a therapeutic drug. The microparticles could have a combined use as a drug delivery agent as well as a diagnostic agent. The microparticles could be combined with other therapeutics, such as small molecule drugs, to make a combination therapy which may address different therapeutic targets or enhance the effect on a single target. The dosing of the therapeutic agent can be altered by altering the composition of the microparticle, or more specifically the amount of miRNA and/or additional drugs that it contains; this can improve the efficacy and/or safety of a therapy. The microparticle can be designed to release the carried microRNA at different times, for example in a delayed release formulation to control the dosing time or reduce potential toxicity of the carried miRNA. The microparticle can be designed to target a specific organ or set of organs, to enhance function where needed and to reduce the possibility of adverse side effects. The microRNA particle can be designed to release its cargo upon a triggered external force, such as by an electromagnetic field or ultrasound wave, as non-limiting examples. The external trigger would then enable the release of the cargo at the time and place desired. The microparticles can be designed to release its cargo dependent on the physiological environment in which it is found, with non-limiting examples being local temperature, pH or ionic strength. The microparticles can also be used to screen for existing or unknown physiological or diagnostic actions of themselves or in combination with other agents.

The present disclosure provides a medicament for treatment of dysregulated immune response, comprising the miRNA-based particles or the compositions disclosed above. The dysregulated immune response may be acute respiratory distress syndrome, or it may be myocardial dysfunction in sepsis or a combination of both.

The dysregulated immune response may be any one or combination of acute kidney injury, acute encephalopathy, hemodynamic instability, liver dysfunction, gut dysfunction, and muscle dysfunction. All of these may contribute to increased morbidity, mortality and post-ARDS, post-cardiomyopathy and post-ICU syndrome.

This treatment is equally applicable to human subjects as well as animal subjects.

### EXAMPLES

Non-limiting exemplary data will be for natural or synthetic miR-193b-5pINH mimic and natural or synthetic miR-187-5p in a 4-lipid carrier, effective to treat cells and mice with two models of lung injury: from viral and bacterial infections. Likely product description for miR-193b-5pINH: a formulation to increase lung surfactant in patients with damaged lungs. Likely product description for miR-187-5p mimic: a formulation to treat cardiomyopathy accompanied by dysregulated immune response.

### 1. miRNA

### Exemplary Data:

### Lipid Nanoparticle:

Referring to FIG. 1, an exemplary lipid nanoparticle (LNP) is shown comprised of four lipids: an ionizable lipid (DLin-MC3-DMA), cholesterol, a structural helper lipid (DSPC), and a PEGylated-lipid (DMG-PEG2000) in a mol % ratio of 50:38.5:10:1.5, respectively used to encapsulate the microRNA (miRNA). Microfluidics were used to control the mixing of the lipid-containing and miRNA-containing solutions. The formulation includes an ionizable cationic lipid that is positively charged in the formulation buffer (in the range about pH 3.5-5.5, and preferably about pH 4), which binds and protects the negatively charged miRNA, but is neutral in the storage buffer (PBS, pH 7.4) When endocytosed by acidic endosomes, it is ionized once again, facilitating endosomal escape.¹⁸ Cholesterol is key in the structure of the LNP, and is important for endosomal release. Cholesterols are non-limiting examples of sterols. The PEGylated lipid stabilizes the structure of the final LNP and is gradually replaced by a protein corona (including opsonins, leading to opsonization). In the formulations studied by the inventors, it is generally thought that the corona is from Apo-E and endocytosis is via the LDL receptor.

Modified ionizable lipid can be accomplished by simple aliphatic epoxide reactions with amines. The carried fractions of cationic lipid can govern which organ is targeted. The LNPs are characterized by e.g., diameter, zeta potential, lipid composition by LC-MSMS, loading, and transfection efficiency (GFP mRNA).

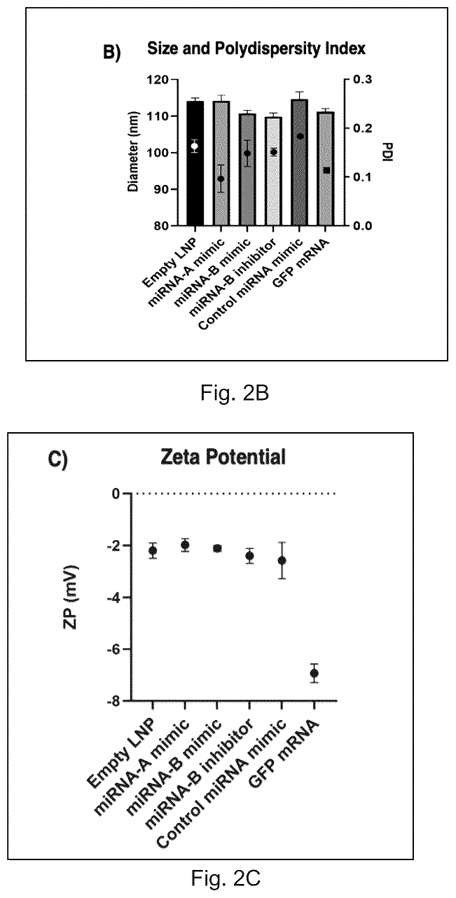

A lipid film containing DLin-MC3-DMA, DSPC, Cholesterol, and DMG-PEG2000 at a 50:10:38.5:1.5 molar ratio was prepared by combining the separate lipid components dissolved in chloroform. For LNP formulation, the aliquots were then dried with inert gas (N₂ or Ar) then placed in a vacuum chamber overnight to remove residual solvent. The lipid film was dissolved in ethanol at a concentration of 6.8 mM (3.6 mg total lipid in 1 mL ethanol). The miRNA solution was prepared by suspending 5 nmol miRNA mimic or inhibitor in 1200 uL 0.1 M acetate buffer (pH 4). The miRNA and lipid solutions were then mixed via microfluidic mixing (NanoAssemblr Benchtop™) with a flow rate ratio of 3:1 respectively, and a total flow rate of 9 mL/min. After mixing, the LNPs were diluted to 12 mL with PBS (pH 7.4) and were purified and by filtration with a 30 kDa cut-off centrifugal filter before resuspension in PBS and storage in a 4° C. fridge. Standardized characterization methods confirm the quality of our LNPs as shown in FIG. 2. There are modest differences between the LNPs by size, polydispersity, zeta potential; encapsulation efficiency will benefit from optimization.

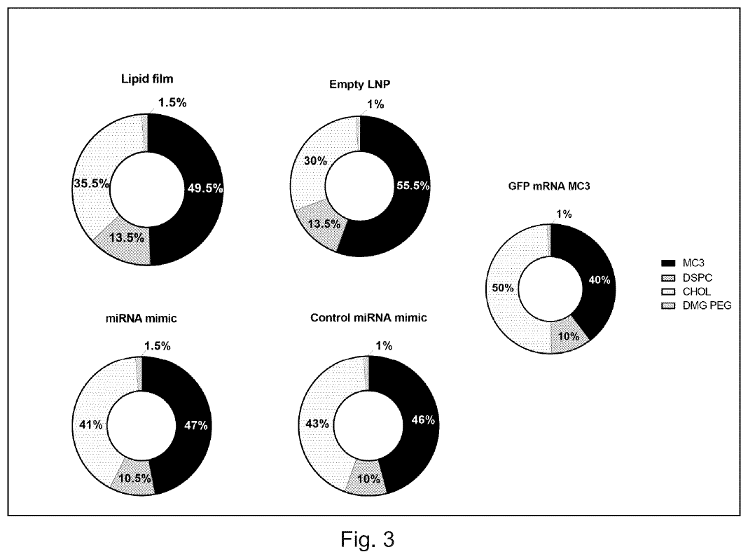

There can be a benefit to increased cholesterol fraction to enhance endosomal release. Typically, only 1 to 2% of LNPs exit the endosome to release their cargo. Increasing the cholesterol and/or changing sterol type (e.g., to β-sitosterol²³) can dramatically increase LNP cargo release and hence transfection efficiency. It has at times been assumed that the LNP component fractions do not change much upon miRNA loading. We have developed a reliable, mass spectrometry method for measuring actual cholesterol (and lipid) components, illustrated by the results shown in FIG. 3, and we see that the change in composition can be significant. Hence, precise preparation and characterization after loading is important.

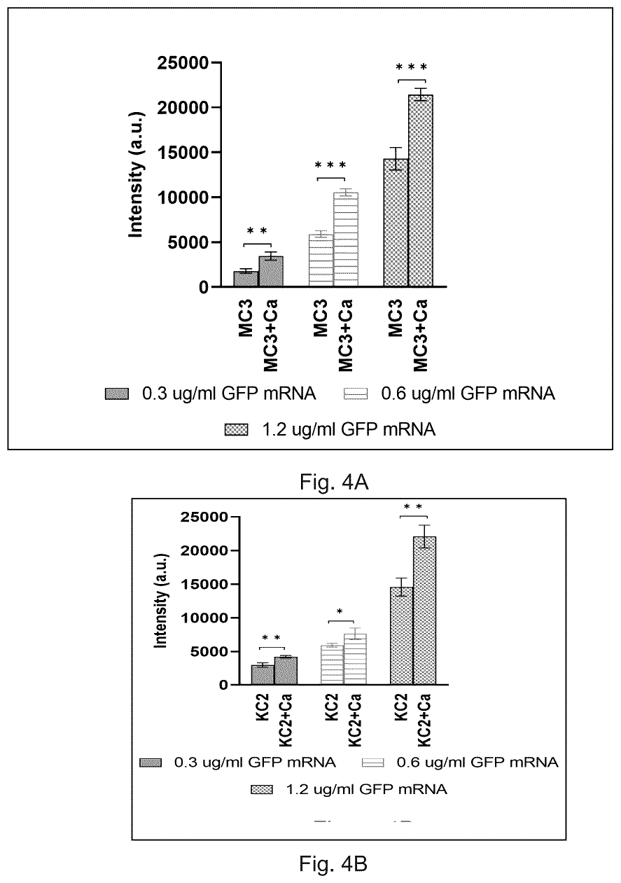

A way to test the value of increased cholesterol fraction is to increase the mol % of cholesterol (and other sterols) and compare LNP uptake with GFP fluorescence from RNA loaded LNP mRNAs. The inventors have discovered that adding divalent ions to the lipid formulation improve transfection. FIGS. 4A and 4B illustrate the value of this approach. It is possible to alter the anionic/cationic lipid ratio to optimize lung targeting. The approach here is distinct from the work of Cheng and coworkers²⁴; microRNA mimics and inhibitors have different constraints on their structures and the characteristic control parameters in LNP loading that the nucleic acids studied and reported on in the Cheng work.

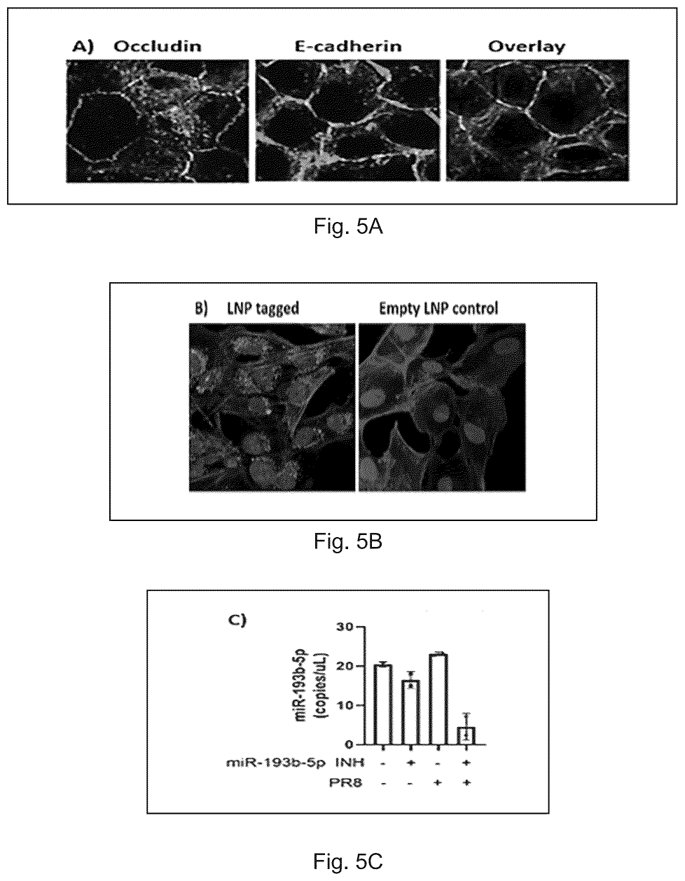

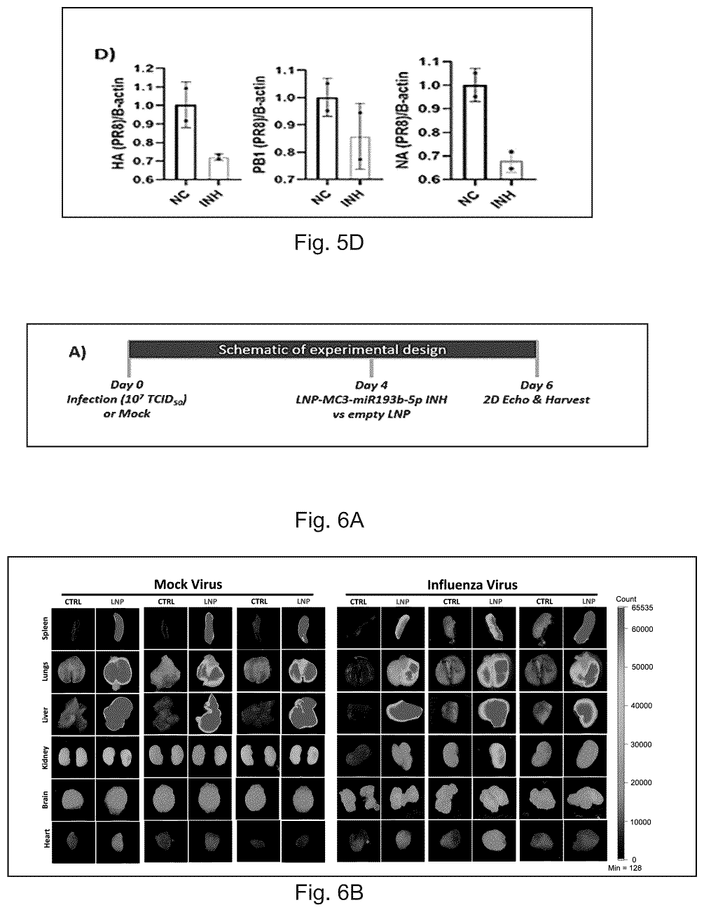

Illustrative data on in vitro tests of formulations of LNPmiR-193b-5pINH are presented next. MiR-193b-5p is expressed in endothelial, epithelial and myeloid derived cells in response to TNF-□ and INF-β. OCln is expressed in endothelial, epithelial and myeloid cells, in murine and human lungs. We have shown uptake of LNPs loaded with fluorescently labelled miRNA mimic and inhibitor in primary human distal bronchial epithelial airway cells (BEAS2b), primary human microvascular endothelial cells (HPMECs) and non-small-cell lung cancer cell line, (CALU-3). All express OCln (FIG. 5A example CALU3). Briefly, viral stocks of influenza A/PR/8/34 (H1N1/PR8) were prepared in MDCK (Madin-Darby canine kidney) cells (viral titers were determined by plaque assay). Cells were exposed to virus (multiplicity of infection MOI=1)±empty LNP (2.7×109 LNPs/ml); LNP containing scrambled/negative RNA control (NC, 10 nM); or LNP miR193b-5p INH (10 nM). FIGS. 5B, 5C show LNPs were able to deliver miR-193b-5p to cells—expression levels were decreased according to digital droplet PCR (ddPCR); and inhibitor had an effect—viral replication was decreased as measured by semi-quantitative PCR (qRT-PCR) for PR8 virus proteins and in FIG. 5C (N=2). In parallel, Jurkat E6.1 (immortalized line of human T lymphocyte) and BEAS2b cells were used to determine cytotoxicity, measured by standard Annexin V/PI and PrestoBlue cell viability.

A non-limiting example of an in vitro H1N1 screen design is as follows. In vitro H1N1 screen design: CALU-3 human lung cells (they make tight junctions in culture) and BEAS2b (express tight junction proteins) are cultured in DMEM-F12 (1:1) medium, and MDCK cells are maintained in DMEM supplemented with 10% fetal bovine serum (FBS) and at 37° C., 5% CO2. Viral stocks of influenza A/PR/8/34 (H1N1) (ATCC: VR-1469) are prepared in MDCK cells and the viral titer are determined by plaque assay. Virus stocks will be diluted with serum-free medium for adsorption. Initial dose-response studies are be done to establish the dose of the selected drug and/or metal that have no impact on CALU-3 cells viability (see cell viability assay).

Subsequently monolayers of CALU-3 cells in six-well plates are adsorbed with H1N1 at multiplicities of infection (MOI) of 0.05, 0.5 or 5 for 60 mins at 37° C., washed three times with PBS, and incubated in medium containing 2% FBS and the compounds of interest at the indicated concentrations. Samples (100 uL/well) at 1, 4, 8, 24, 48 and 72 hrs and supplemented with the same volume of media containing the corresponding compound concentrations. Collected media are centrifuged at 20,000×g for 1 min, and the virus production in culture supernatants is be determined by the plaque assay. Plaque forming units (PFU) is determined by plaque assay using six-well plates. In brief, MDCK monolayer cells are be adsorbed with 10-fold serial dilutions of the supernatant and overlaid with 1:1 mixture of 1.8% low melting point agarose and 2× viral growth medium (2×VGM, 2×DMEM containing 6 mg/ml bovine serum albumin and 2 mg/ml TPCK-Trypsin). The cells are fixed with 10% formaldehyde solution after 72 hr incubation and plaques will be detected by staining with 0.1% crystal violet in 20% methanol.

### Cell Viability Assay

CALU-3 monolayer cells are seeded in 96-well plates and adsorbed with H1N1 at a MOI of 5 or the same volume of 1×VGM for 60 min, washed three times with 1×PBS, and incubated in the medium containing 2% fetal bovine serum, and the compounds of interest. To determine the effects of drug exposure on anti-viral activity, cells are incubated with the treatments for 1, 4, 8, 24, and 48 hrs. At the indicated time points, the treatments are removed and the wells are replenished with medium supplemented with 2% FBS. Cell viability are measured based on membrane integrity where all cells are stained with Hoechst3334241 and non-viable cells with ethidium homodimer-I at 72 hr post-infection. The cells are incubated for 20 minutes at 37 C and imaged using the INCell Analyzer 2200 platform (GE Healthcare) and cell counts can be performed using Developer Toolbox 1.9 software (GE). The EC50 can be calculated using viable cell counts. A no infection control should be included to determine CC50. We found the viability improved for viral infected cells upon treatment with LNPs.

Accordingly, a preferred route of administration is IV, even if efficient delivery to the lung may be limited by biodistribution or/and removal of the active drug by other organs. Ultrasound may be viewed as an alternative to guide delivery of effective therapeutics across edema-fluid filled alveoli, as a non-limiting example, a decafluorobutane microbubble, which can exhibit a net positive surface potential, could be integrated with lipid nanoparticles imagined as above, if a negatively charged lipid is included in the outer layer of the lipid nanoparticle.

### Exemplary Data on miRNA Delivery Using Microfluidics Loaded Lipid Nanoparticles

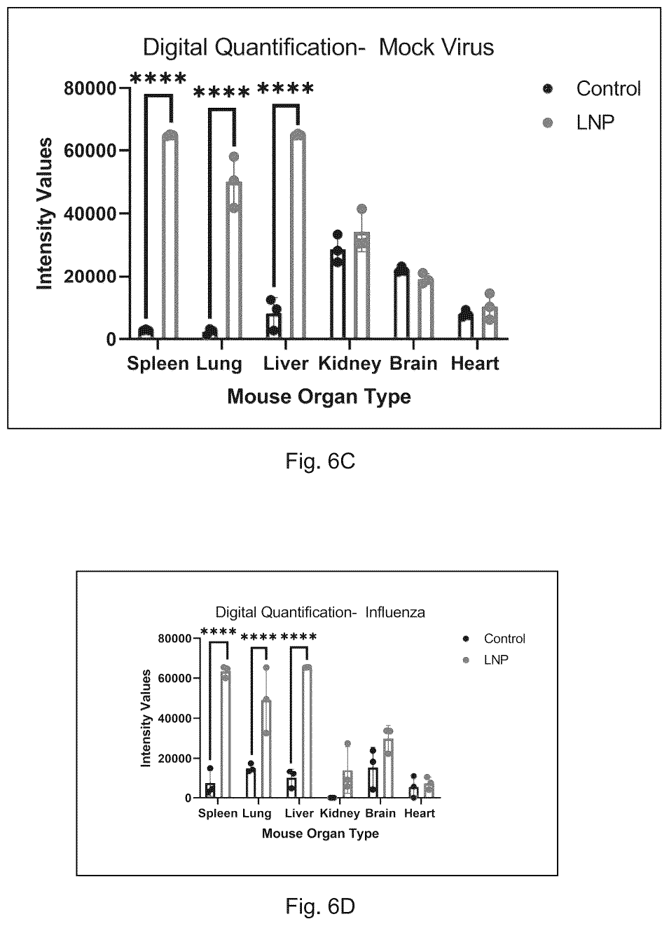

We show non-limiting exemplary data on in vivo delivery of LNP-miR-193b-5p INH. We delivered empty fluorescent LNPs or LNPs loaded with labelled miRNA by nasopharyngeal, intratracheal instillation, aerosolization, or intravenous routes of administration at 6, 12, 24, 48, 72, 96 or 120 hours. Intra-nasal delivery, resulted in increased fluorescence in naso- and oropharynx (little went to lung, not shown). When correlating with histology, both intratracheal and aerosolized LNPs did go to lung but were preferentially found in non-injured areas. Intravenous administration demonstrated LNPs in severely injured areas of the lung. We cut cryosections and were able to visualize labelled miRs in severely damaged alveolar spaces (up to 96 hrs), using miRNAscope in situ hybridization (data not shown). Labelled LNPs and miR-INH were detected in extrapulmonary organs, (especially spleen, and liver FIGS. 6B to 6D), associated with preliminary evidence of decreased inflammatory gene expression in these organs (data not shown), suggesting an added benefit to extra-pulmonary organs by systemic deliver. Fluorescent LNPs, or labelled miRNA, were not detected beyond 96 hrs following injection.

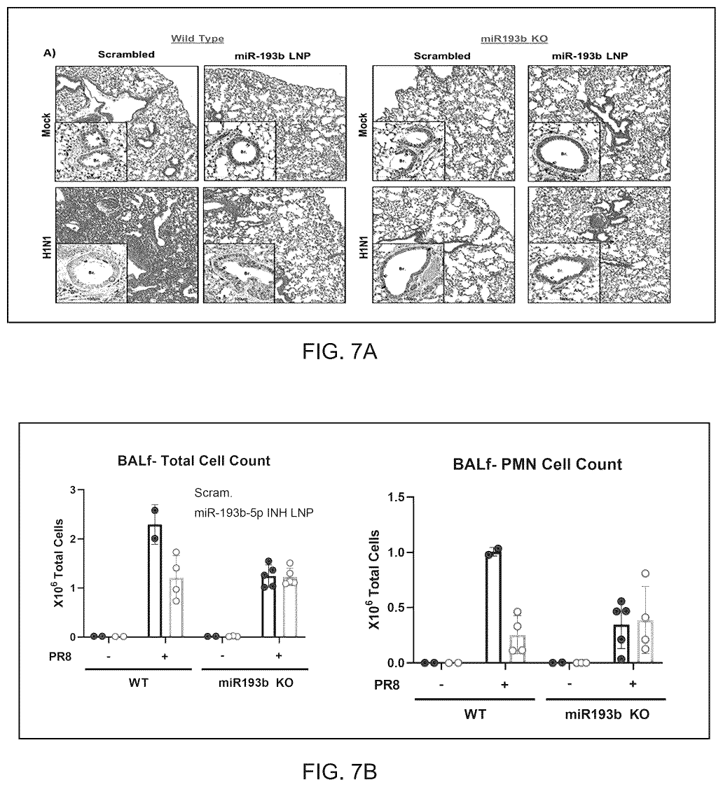

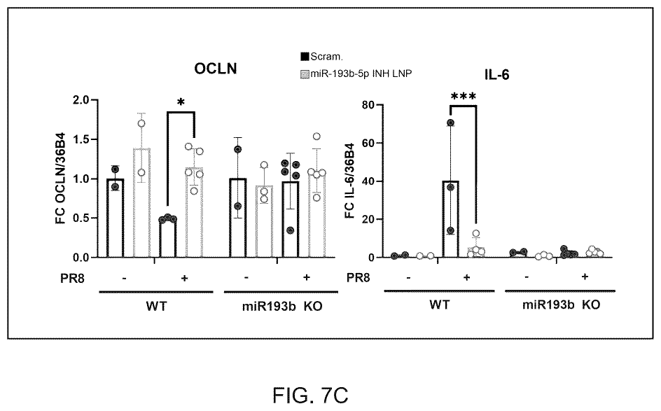

MiR-193b-5p expression was decreased as shown by digital droplet PCR (ddPCR) in samples harvested 48 hrs after LNPmiR-193b-5INH administration (i.e. day 6 post infection). MiR-193bKO and wild-type (WT) litter mates (8-12 weeks) were intranasally (IN) infected with H1N1 (PR8 strain A/Puerto Rico/8/1934). Infection with a median tissue culture infective dose [TCID] of 107 results in 90-100% mortality by day 7. Mice develop progressive weight loss, hypothermia, and arterial hypoxemia. Four days post infection mice were randomized to receive an equal volume (100 ul) intravenous (IV): (i) empty LNP (6.3E11/mouse), (ii) LNP scrambled negative control (NC 0.4 nmol/mouse); or (iii) LNP miR-193b-5p inhibitor ([INH], 0.4 nmol/mouse). Mice were sacrificed at day 6 for (a) assessment of lung injury (histology), inflammation (BALf total cell count, PMN extravasation and IL-6 expression) and effect on target gene OCln expression (FIGS. 7A to 7C). These data strongly suggest inhibition of miR-193b reduces virus induced lung injury compared to a scrambled control; the empty LNPs were not associated with overt increase in lung inflammation in WT or miR-193bKOs. Exemplary test formulations of LNPmiR-193b-5pINH in H1N1 mouse models of influenza infection have been tested.

In vivo outcome measures that support the assertion of efficacy include survival. The mice are monitored for morbidity as evidenced by weight loss, clinical signs of disease including lethargy, ruffled fur, hunched back, labored breathing and hypoxemia (pulse oximetry). Detailed assessment of lung injury will occur include as non limiting examples histology, lung injury scores (LIS), BALf (total cell count, percent neutrophilia and myeloperoxidase (MPO) activity assay), and mediators in BALf and lung tissue lysates using a pre-made murine specific Cytometric Bead Array. It is useful to assay Th1 and Th2 responses. Membrane permeability can be determined by measuring total protein, Evans Blue Dye, and IgM leak in BALf. Viral load can be quantified by plaque forming units [PFU]/ml, HAU, immunohistochemistry for PR8 antigen in lungs, Western blot for viral NP and qRT-PCR for viral transcripts: hemagglutinin (HA), neuraminidase (NA) and polymerase (PB1). One can determine cell-specific regulation of miR-193b-5p by in situ hybridization in lung tissues using Basescope with immunostaining for cell-specific markers (CK18 (epithelial cell marker) or/and CD31 (endothelial cell marker)) by immune-fluorescence or brightfield immunohistochemistry to detect pre-miR-193b, -3p, -5p, and Occludin (probes for mmu-pre-miR-193b, -5p and -3p). Immunoreactivity can be analyzed by determining the integrated optical density (IOD) per stained area in μm2, using Image-Pro Plus software. Where appropriate, in addition to measuring miR-193b-5p and -3p by qRT-PCR, one can determine function by assessing expression levels of target mRNAs and off target effects by RNA sequencing analysis as previously published. Total RNA from WT and KO mice infected with PR8 and treated with the miR-193b-5p INH, mimic (MIM) or respective control oligonucleotides can be hybridized to RNAseq arrays.

### Exemplary In Vitro Tests of Formulations of LNPmiR-193b-5p-INH

MiR-193b-5p is expressed in endothelial, epithelial and myeloid derived cells in response to TNFα and INFβ (²⁵ and ²⁶). OCln is expressed in endothelial, epithelial and myeloid cells, in murine and human lungs. We have shown uptake of LNPs loaded with fluorescently labelled miRNA mimic and inhibitor in primary human distal bronchial epithelial airway cells (BEAS2b), primary human microvascular endothelial cells (HPMECs) and non-small-cell lung cancer cell line, (CALU-3). All express OCln (FIG. 5A example CALU3). Briefly, viral stocks of influenza A/PR/8/34 (H1N1/PR8) were prepared in MDCK (Madin-Darby canine kidney) cells (viral titers were determined by plaque assay). Cells were exposed to virus (multiplicity of infection MOI=1)±empty LNP (2.7×10⁹ LNPs/ml); LNP containing scrambled/negative RNA control (NC, 10 nM)^{27,4}; or LNP miR193b-5p INH (10 nM) as published.

FIGS. 5B and 5C shows LNPs were able to deliver miR-193b-5p to cells—expression levels were decreased by digital droplet PCR (ddPCR); and inhibitor had an effect—viral replication was decreased as measured by semi-quantitative PCR (qRT-PCR) for PR8 virus proteins as described in references (²⁵ and ²⁶) (N=2). In parallel, Jurkat E6.1 (immortalized line of human T lymphocyte) and BEAS2b cells were used to determine cytotoxicity, measured by standard Annexin V/PI and PrestoBlue cell viability.

### Exemplary In Vivo Delivery of LNP-miR-193b-5p-INH

The inventors delivered empty fluorescent LNPs or LNPs loaded with labelled miRNA by nasopharyngeal, intratracheal instillation, aerosolization, or intravenous routes of administration at 6, 12, 24, 48, 72, 96 or 120 hours. Intra-nasal delivery, resulted in increased fluorescence in naso- and oropharynx (little went to lung, not shown). When correlating with histology, both intratracheal and aerosolized LNPs did go to lung but were preferentially found in non-injured areas. Intravenous administration demonstrated LNPs in severely injured areas of the lung. We cut cryosections and were able to visualize labelled miRs in severely damaged alveolar spaces (up to 96 hrs), using miRNAscope in situ hybridization (data not shown). Labelled LNPs and miR-INH were detected in extrapulmonary organs, (especially spleen, and liver FIGS. 6B, 6C and 6D), associated with preliminary evidence of decreased inflammatory gene expression in these organs (data not shown), suggesting an added benefit to extra-pulmonary organs by systemic deliver.

Fluorescent LNPs, or labelled miRNA, were not detected beyond 96 hrs following injection. MiR-193b-5p expression was decreased as shown by digital droplet PCR (ddPCR) in samples harvested 48 hrs after LNPmiR-193b-5INH administration (i.e., day 6 post infection). MiR-193bKO and wild-type (WT) litter mates (8-12 weeks) were intranasally (IN) infected with H1N1 (PR8 strain A/Puerto Rico/8/1934²⁸). Infection with a median tissue culture infective dose [TCID] of 10⁷ results in 90-100% mortality by day 7. Mice develop progressive weight loss, hypothermia, and arterial hypoxemia²⁹. Four days post infection mice were randomized to receive an equal volume (100 ul) intravenous (IV): (i) empty LNP (6.3E11/mouse), (ii) LNP scrambled negative control (NC 0.4 nmol/mouse); or (iii) LNP miR-193b-5p inhibitor ([INH], 0.4 nmol/mouse). Mice were sacrificed at day 6 for (a) assessment of lung injury (histology), inflammation (BALf total cell count, PMN extravasation and IL-6 expression) and effect on target gene OCln expression (FIGS. 7A to 7C). The present data strongly suggests inhibition of miR-193b reduces virus induced lung injury compared to a scrambled control; the empty LNPs were not associated with overt increase in lung inflammation in WT or miR-193bKOs.

FIGS. 7A to 7C show LNPmiR-193b-5pINH systemic administration at day 4 post H1N1/PR8-infection mitigates injury in WT mice without overt harm to MIR-193bKOs. We have performed approx. dose finding, and administration timing experiments (data not shown) IV delivery of LNP-miR-193b-5pINH, 6 hrs post-cecum ligation and puncture (CLP), resulted in a trend towards decreased inflammatory cell infiltration, edema formation and histological lung injury (data not shown). The data shows a trend towards decreased bacterial counts and sepsis-induced cardiomyopathy, with preserved ejection fraction, fractional shortening, and alpha (α)-myosin heavy chain (Myh6) expression^{30,31} and decreased TNF and S100A8 (important in injured hearts) expression compared to LNPs alone or LNP-NC, suggesting decreased organ injury (data not shown).

### Discovery and Demonstrated Roles of miR-193b-5p and miR-187-3p in ARDS/Acute Lung Injury

(i) A primer on ARDS/Acute Lung Injury is Available in Reference³².

The inventors previously reported⁴ that miR-193b-5p binds to the 3′-UTR of the mRNA of Occludin (a main component of tight junctions) and Surfactant Protein C (a critical protein involved in reducing surface tension) leading to post-transcriptional degradation and decreased protein expression. Inhibition of miR-193b-5p is salutary (reduces lung injury) in 3 models of acute lung injury—a model of sterile sepsis (endotoxemia caused by instillation of lipopolysaccharide to the lung—this is a model of direct lung injury); cecum ligation and puncture (a model of sepsis-induced ARDS—secondary extrapulmonary ARDS and an unreported result), and a model of influenza A virus induced lung injury and an unreported result. Preventing occludin degradation in our murine models of ARDS results in: decreased alveolar-capillary barrier disruption, neutrophil infiltration, inflammatory cytokine and chemokine expression, decreased bacterial and viral load and improved survival.

The inventors further discovered, and it is previously unreported, that absence of miR-193b-5p protects murine lungs from loss of surfactant C during viral ARDS. Preservation of surfactant is associated with decreased Picrosirius red staining (staining methods for collagen and amyloid) suggesting decrease ARDS associated fibrosis in mice that lack miR-193b-5p.

(ii) Predicted (Based on Theoretically Computed Gene Targets Found in Tarbase v8.0)—Role of miR-193b in Acute Lung Injury:

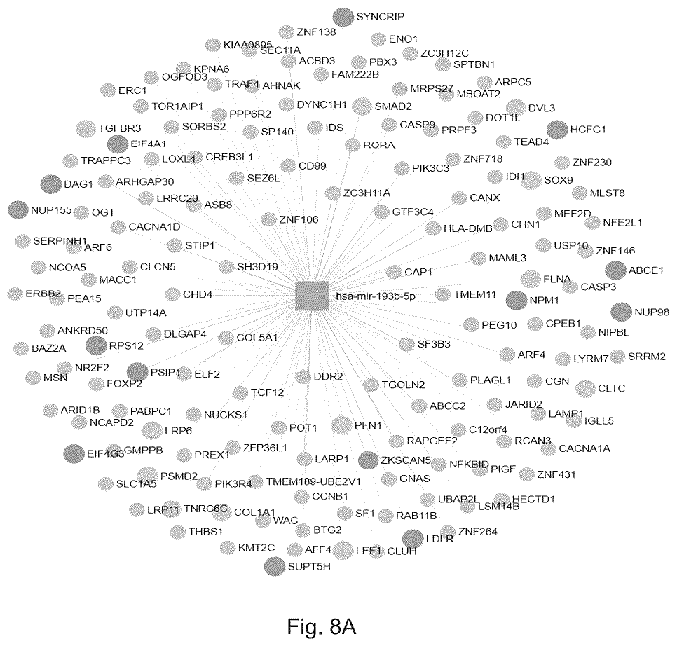

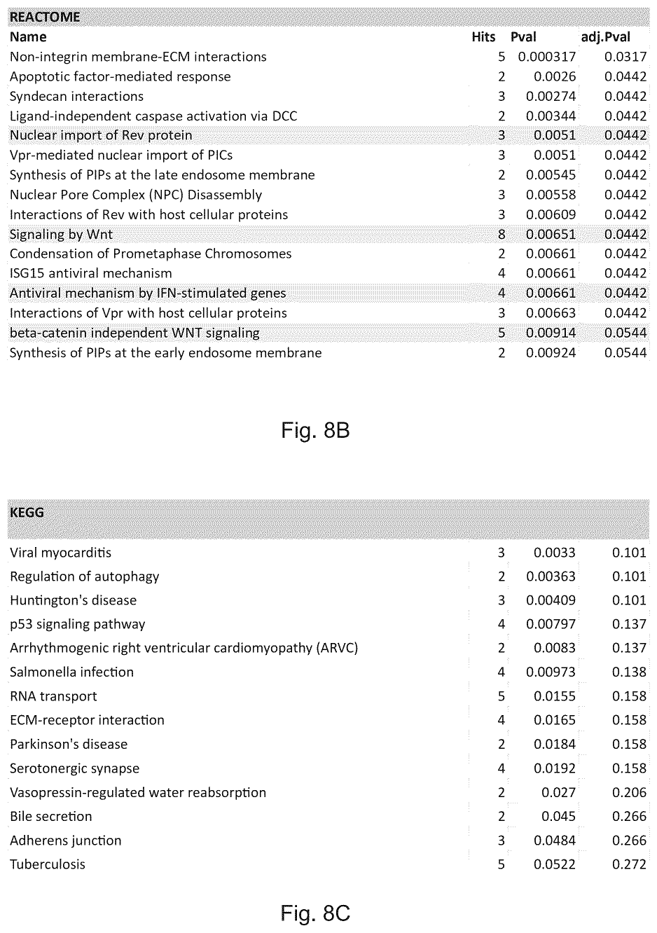

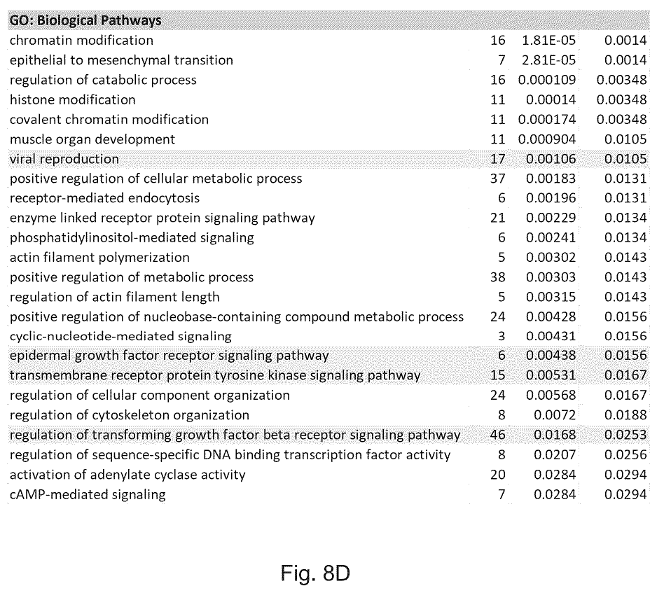

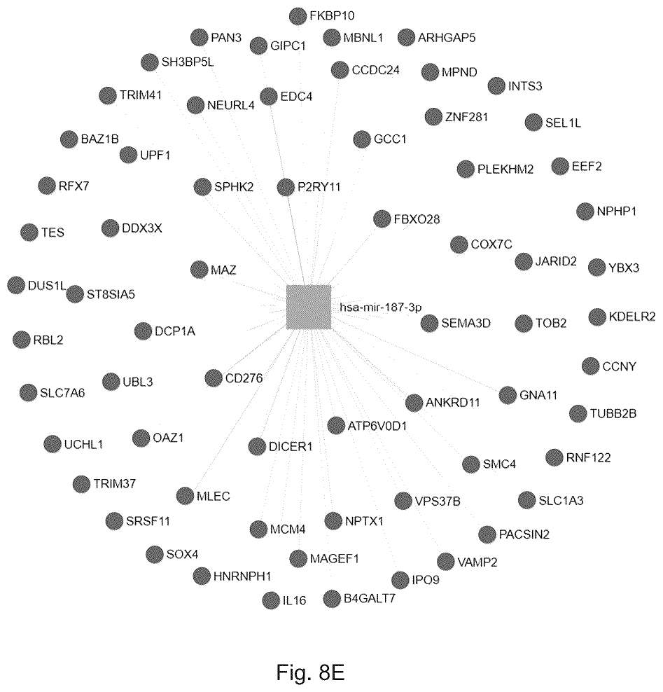

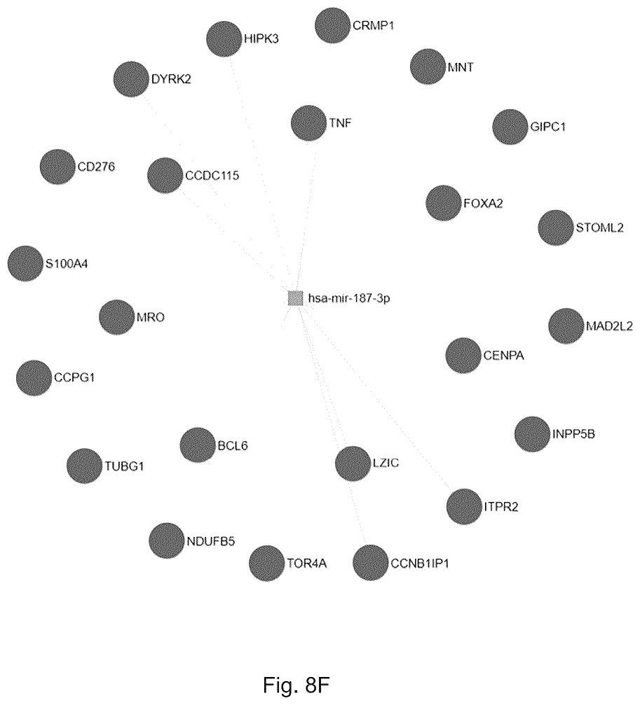

Theoretically computed targets of miR-193b-5p and putative pathways that are affected by miR-193b-5p inhibition are illustrated in FIGS. 8A and 8B. Antiviral (red); fibrosis (TGFb/Wnt pink). HUGO gene symbols are shown for each node for known gene targets of miR-193b-5p. Inhibition of miR-193b-5p is expected to (i) decrease viral load by decreasing the expression of viral replication associated genes and decrease pulmonary fibrosis by decreasing the activation of TGFb/Wnt/Beta catenin related pathways.

(iii) Discovery/Demonstrated—miR-187-3p Mimic

The inventors have demonstrated in vivo and in vitro that miR-187-3p replacement therapy—using a miR-187a-3p mimic transfected into primary neonatal cardiomyocytes and further showed regulation of miR-187a-3p and target genes involved in sepsis-induced heart dysfunction (Itpkc, Lrrc59, and Tbl1xr1). Tbl1xr1 (transducin β-like 1 X-linked receptor 1) is essential in the activation of Wnt-β-catenin—inhibition of canonical Wnt signaling prevents heart failure. In addition, TNF-α, IL-6, and IL-12p40 (important pro-inflammatory mediators in sepsis) are published targets levels are known to have their levels modulated by miR-187a-3p in monocytes, but it has not been known whether they are direct targets in cardiac cells, and whether mir-187-3p binds the 3-UTR of the protein mRNA of IL-6 and IL-12p240. TarBase, a database of computationally supported miR-targets, showed intercellular adhesion molecule-1 (ICAM-1), Jumonji And AT-Rich Interaction Domain Containing 2 (Jarid2), and S100 calcium binding proteins A1 (S100A1) and A4 (S100A4)—involved in myocardial dysfunction. We have discovered a previously unknown role for microRNA-187-3p (miR-187a-3p) and its target genes S100 calcium binding proteins A1 (S100A1) and Jumonji And AT-Rich Interaction Domain Containing 2 (Jarid2) in sepsis and sepsis-induced myocardial dysfunction.

(iv) Predicted (Based on Theoretically Computed and Experimentally Verified Gene Targets in miRTarbase v8.0)—Role of miR-187-3p

MiR-187-3p is less studied and there are less known targets (Tarbase 8.0 and miRTarbase v8.0), as shown in FIGS. 8E and 8F. We have identified miR-187a-3p, and determined a therapeutic mechanism of action involving post-transcriptional inhibition of proinflammatory mediators TNF-α, interleukin 6 (IL6) and the cardiac alarmin S100A1. The latter two are not found in FIGS. 8E and 8F. Novel promising miRs have also been identified for further testing. Exemplary data is provided in support for the central claims.

### Micro-RNA-Based Therapeutics for Sepsis-Induced Myocardial Dysfunction: Exemplary Data

To identify miR-biotargets for the treatment of myocardial dysfunction in sepsis^{33, 34,35} a clonally expanded and immortalised human mesenchymal stromal cell line (ciMSC³⁶)—a source of therapeutically relevant, homogeneous, and reproducible extracellular vesicles (EV) was prepared^{37, 38, 39,40}. Preclinical studies demonstrated marked reduction in inflammation, organ injury, mortality as well as increased bacterial killing after intravenous MSC administration.^{41, 42,43} EV content—mRNAs, microRNAs, lipids and proteins—are thought to impart paracrine biological activity of MSCs.^{44, 45, 46, 47, 48,49} Administration of EVs derived from ciMSCs confers neuroprotection in models of brain injury⁵⁰ and models of graft versus host disease³⁶. Given the immunomodulatory properties of ciMSC-EVs, we tested whether these same EVs, were also able to prevent left ventricular dysfunction in a preclinical model of sepsis contribution of S100A1 to sepsis-induced cardiomyopathy.

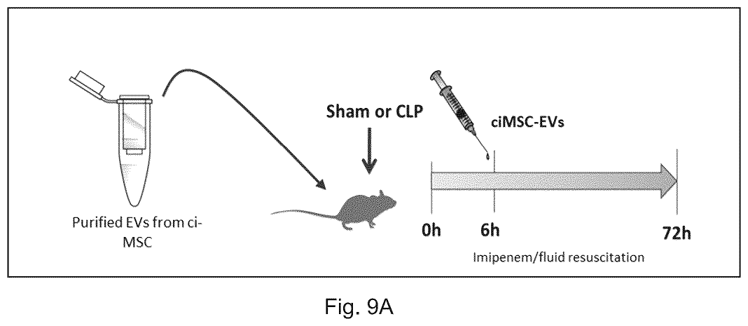

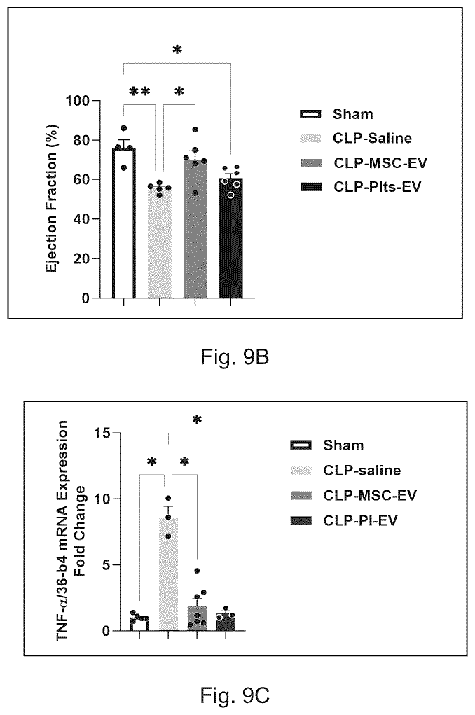

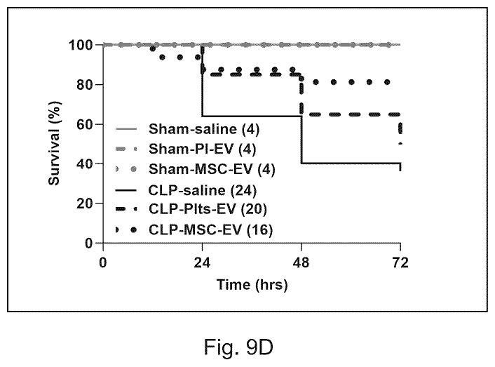

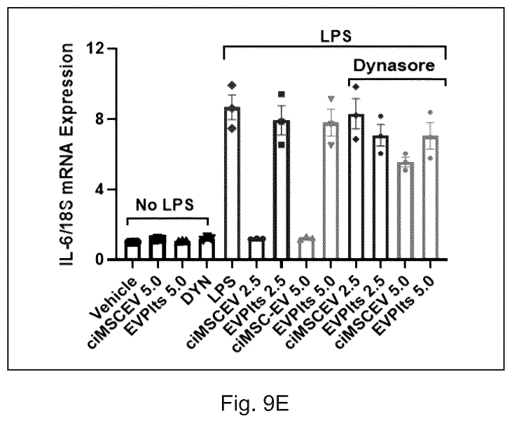

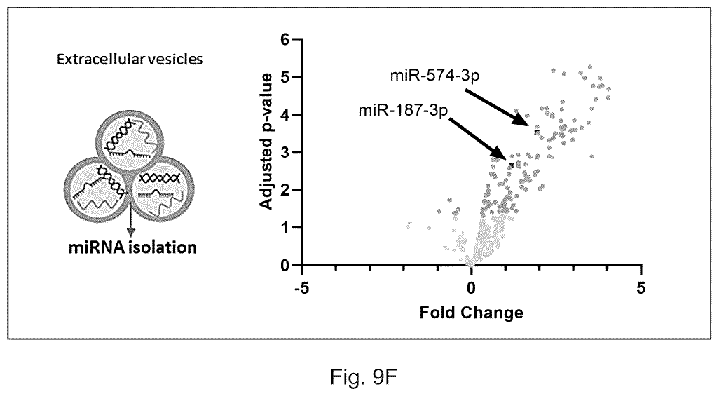

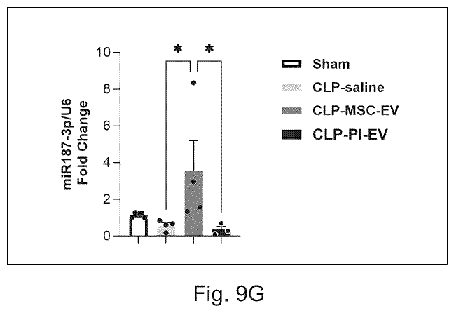

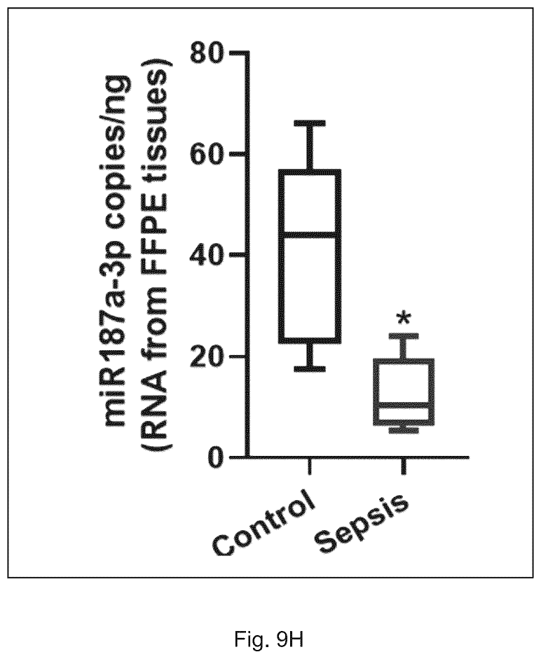

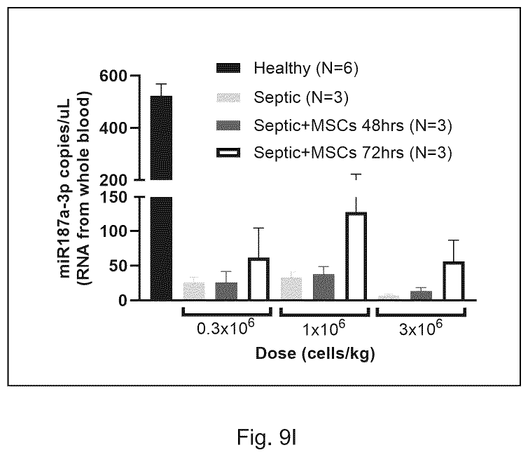

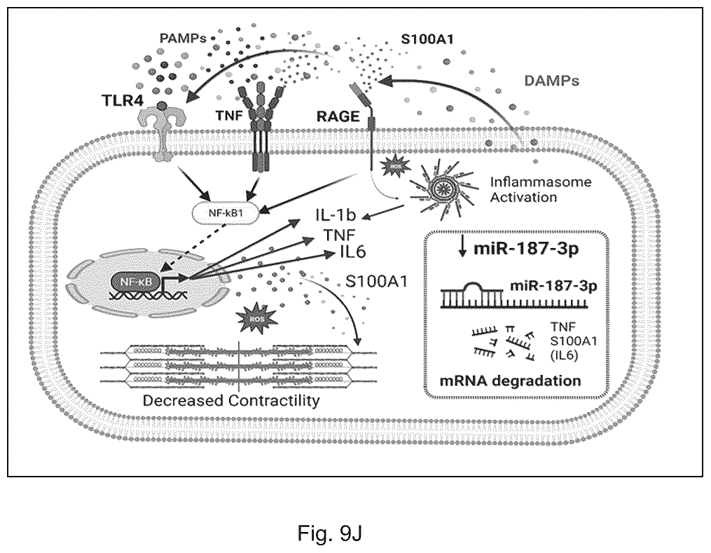

FIGS. 9A-9I present exemplary data and a schematic of the mechanism of action (MoA) for miR-187-3p. FIG. 9A shows a schematic of in vivo experiment. Male and female C57bl/6 mice (12-14 weeks) randomized to CLP or sham surgery received antibiotics (imipenem) and fluid resuscitation (saline). Mice were further randomized to EV preparations from ci-MSCs or platelets (5×10E4 cell equivalents/g BW) delivered IV 6 hrs post-CLP and assessments made at 72 hrs post-CLP. FIG. 9B shows the mean±SEM of percent change in echo-derived Ejection Fraction and FIG. 9C shows TNFa in relation to 36-b4, *p≤0.05; **p≤0.01. FIG. 9D shows the probability of survival at 72 hrs post-CLP, *p≤0.05, **p≤0.01 as shown in FIG. 9E, illustrating the effect on neonatal cardiomyocytes (2×10E5/cells) that were exposed to LPS (10 mg/mL×24 hrs) with ciMSC vs platelet EVs (dose either 2.5× or 5×) with or without Dynasore (DYN, inhibitor of endocytosis). Bars are mean±SEM of IL-6/18S, *p<0.05 vs. vehicle, N=3. FIG. 9F shows results for two independent replicates of ci-MSC and platelet EVs that were sent for miRNA sequencing (HTG Molecular Diagnostics). Volcano plot was used to visualize 66 miRs found to be enriched in ciMSC-EVs compared to platelet EVs (False discovery rate<0.01; Fold change>2). MiRs 187-3p and 574-3p were identified as top enriched miRs in EVs from ciMSCs. FIG. 9G shows miR-187-3p expression increased in hearts from septic mice that received ciMSC-EVs. FIG. 9H shows box plots of miR-187-3p/U6 levels from formalin fixed paraffin embedded post-mortem human hearts from patients who died with sepsis versus non-septic. Data is median±IQR, *p<0.05 vs. non-septic, N=6.

FIG. 9I shows miR-187-3p copy number in whole blood from 9 patients enrolled in the Cellular Immunotherapy for Septic Shock Trial, symbols are values from individual patients, N=3/group) and healthy controls (N=6). RNA was isolated from whole blood collected at baseline, 24, and 72 hrs post MSC infusion. MSC dose (0.3, 1, or 3 million cells/kg). FIG. 9J provides a schematic of MoA for miR-187-3p. miR-187-3p is reduced during sepsis. miR-187-3p decreases the expression of TNFa, and we postulate IL-6 and S 100AI, post-transcriptionally (binds to 3′UTR leading to mRNA degradation), mitigating both Pathogen and Damage Associated molecular pattern receptor stimulation mitigating inflammation and myocardial dysfunction.

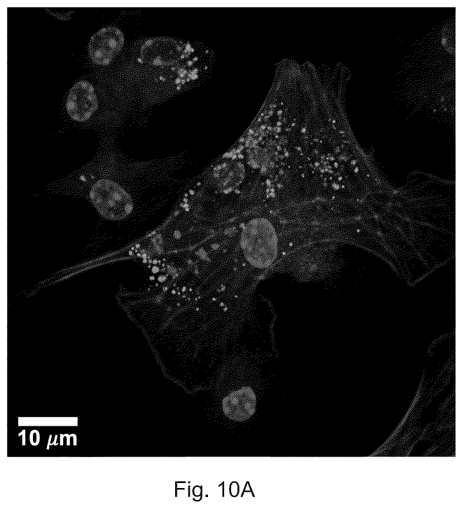

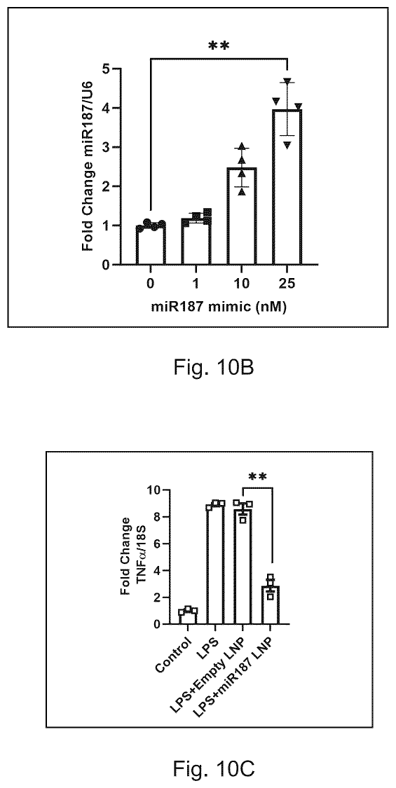

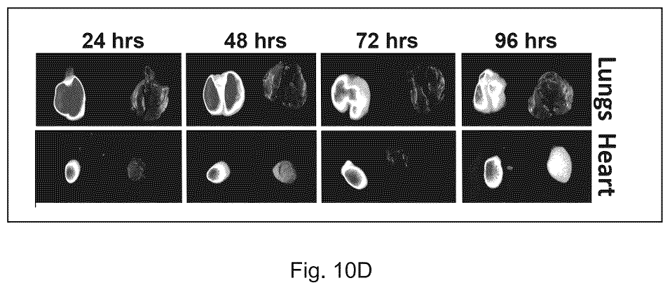

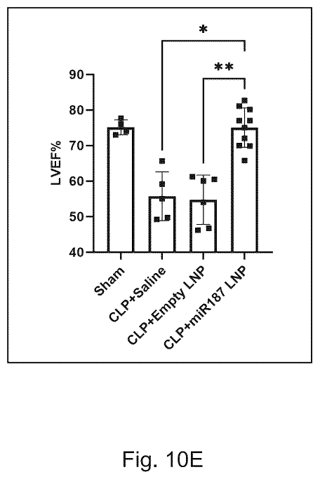

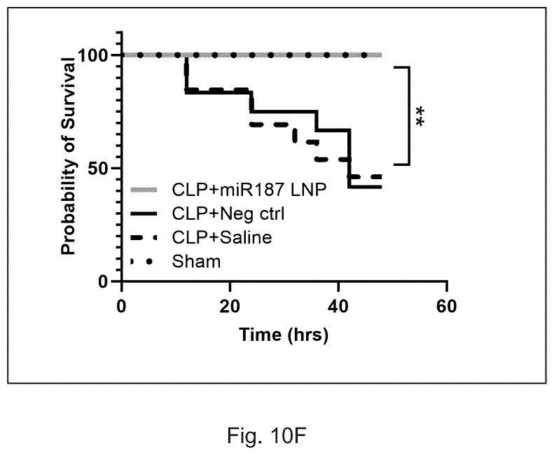

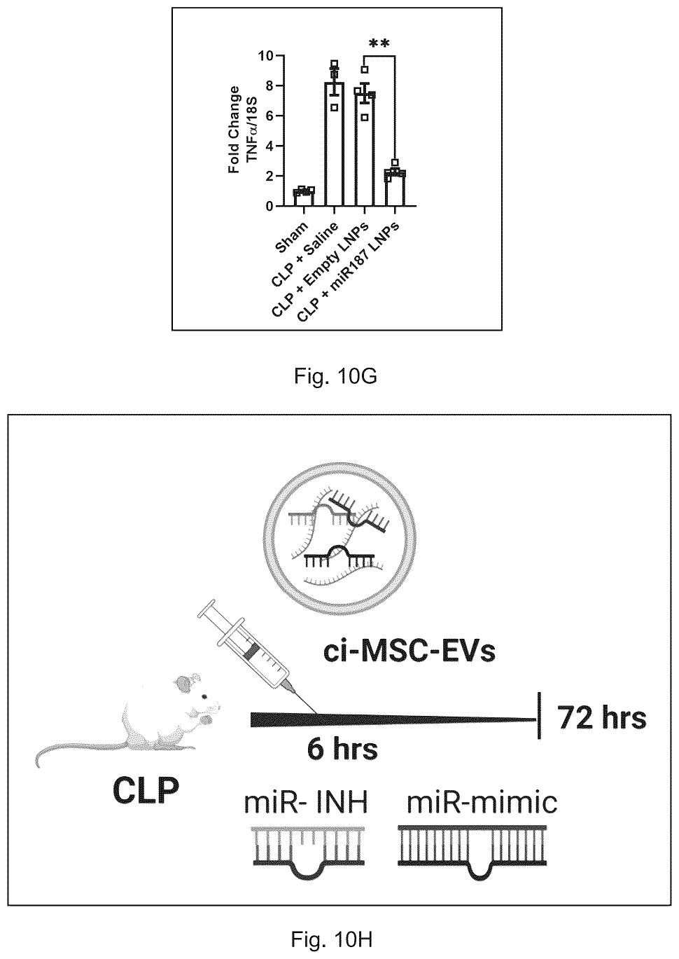

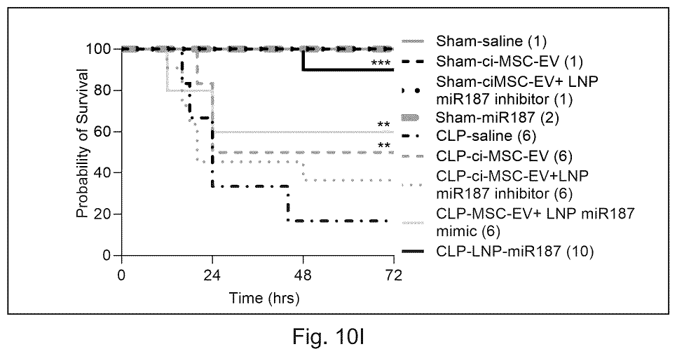

FIGS. 10A to 10I Illustrate that delivery of LNP carrying miR-187-3p attenuates inflammatory gene expression, organ dysfunction and mortality. In vitro: FIG. 10A, an immunofluorescent image, shows uptake of LNPs (green fluorescence) by primary murine neonatal cardiomyocytes (red fluorescence) loaded with 10 mM of miR-187-3p mimic. FIG. 10B shows mean±SEM miR187-3p/U6 24 hrs after LNP-miR-187-3p delivery, n=3. FIG. 10C shows mean±SEM of TNFa/18S in response to LPS (mg/mL)±LNP-miR-187-3p for 24 hrs, n=4. In vivo: Delivery of exogenous miR-1873p to septic mice was examined. LNPs tagged with Cy5 were injected intravenously (IV) in C57bl/6 mice. FIG. 10D provides representative immunofluorescent images show LNP uptake in heart, lung, and spleen. FIG. 10E provides mean+SEM of percent left ventricular (LV) ejection fraction (EF) and fractional shortening (FS), vs. CLP, **p≤0.001 vs. CLP+empty LNP; FIG. 10F shows probability of survival at 48 hrs post-CLP in mice that received LNP-miR-187-3p (n—11) compared to saline (n—13) and empty LNP (n—12); *p≤O.05 vs. CLP. FIG. 10G shows mean±SEM of TNFa/18S in healthy tissue 48 hrs after CLP *p≤0.05, **p≤0.01 (n—3-10). FIG. 10H provides a schematic of a reconstitution experiment. Mice were randomized to receive ciMSC-EVs 6 hrs post CLP with either the miR-187-3p inhibitor or the miR-187-3p mimic. FIG. 10I shows the probability of survival, Log rank test *p≤0.05, **p≤0.01, ***p≤0.001 compared to CLP treated with saline.

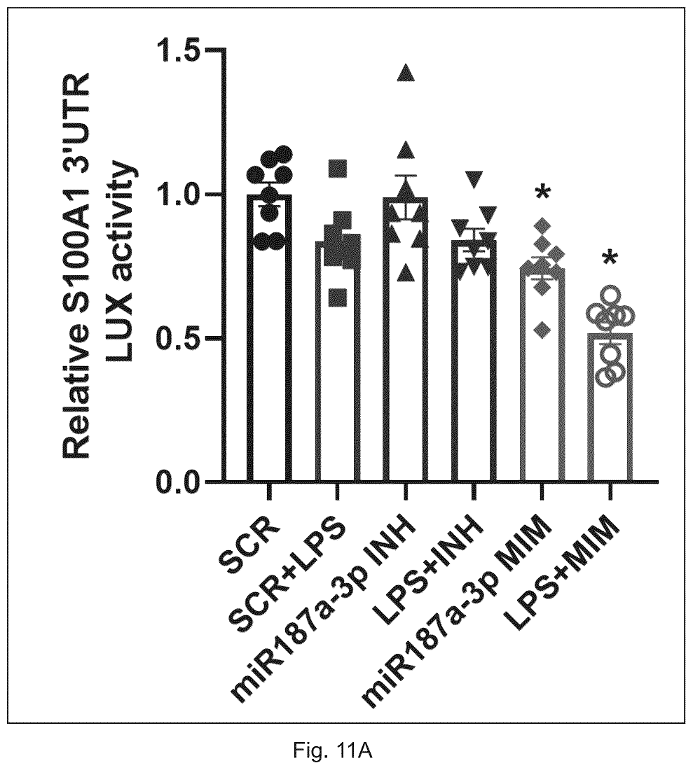

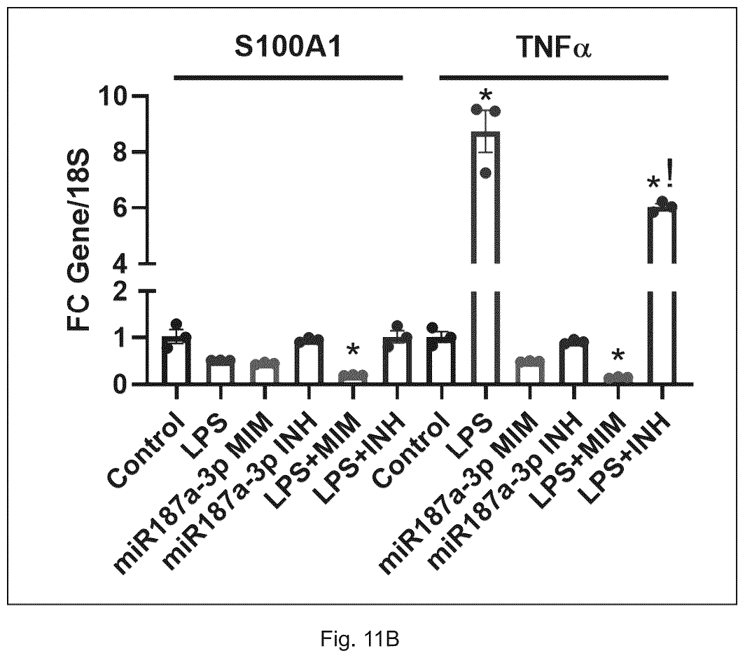

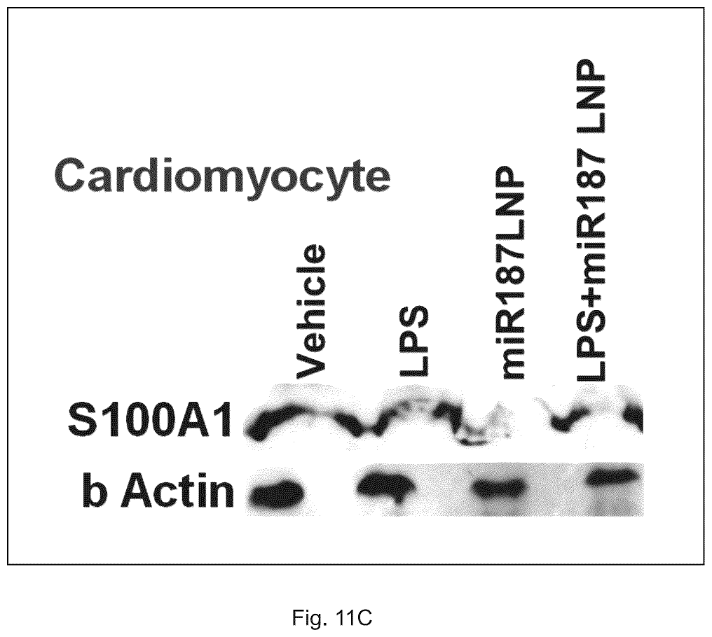

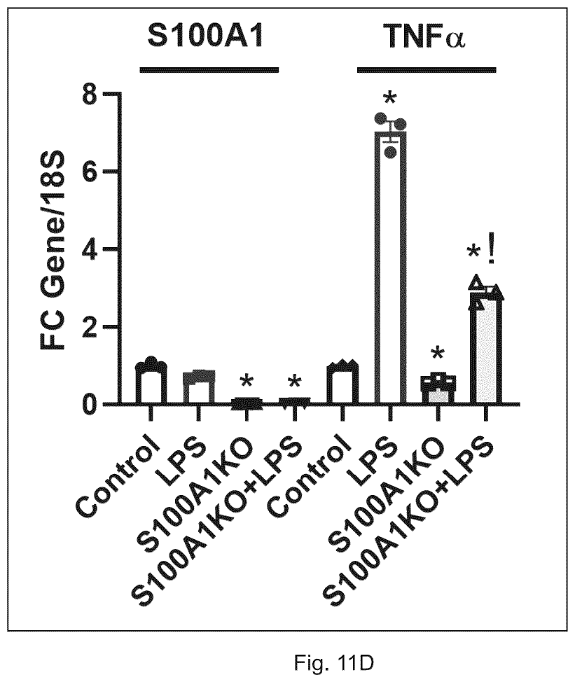

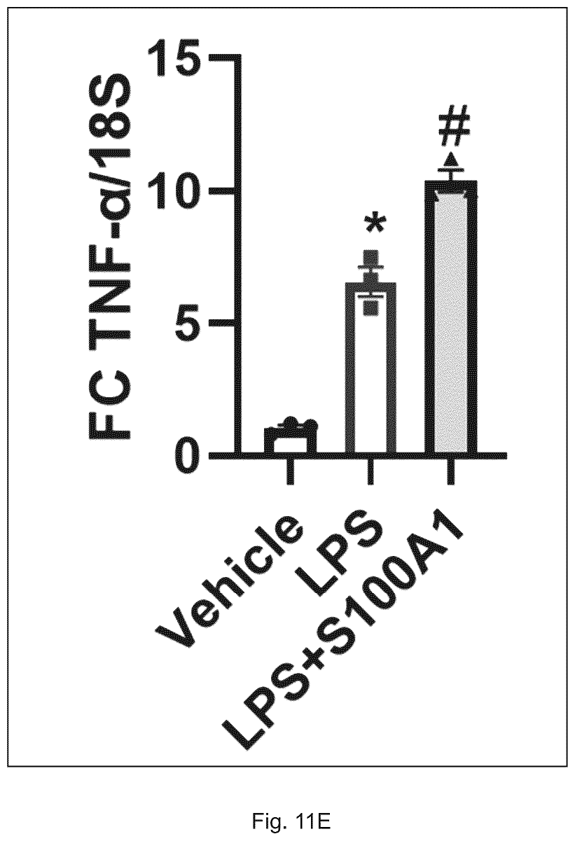

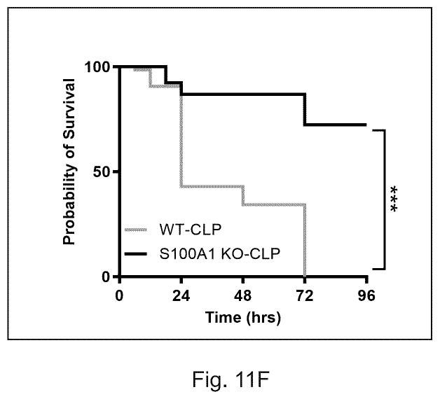

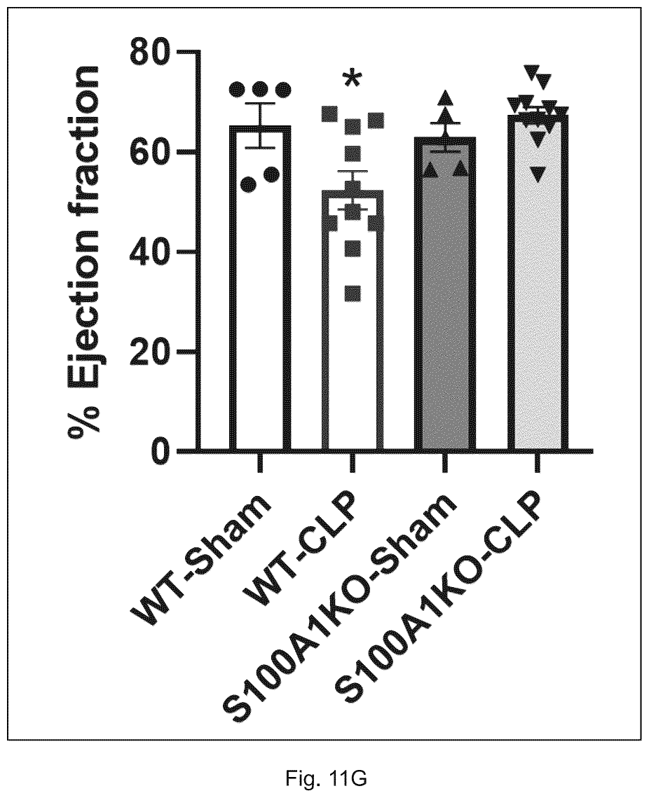

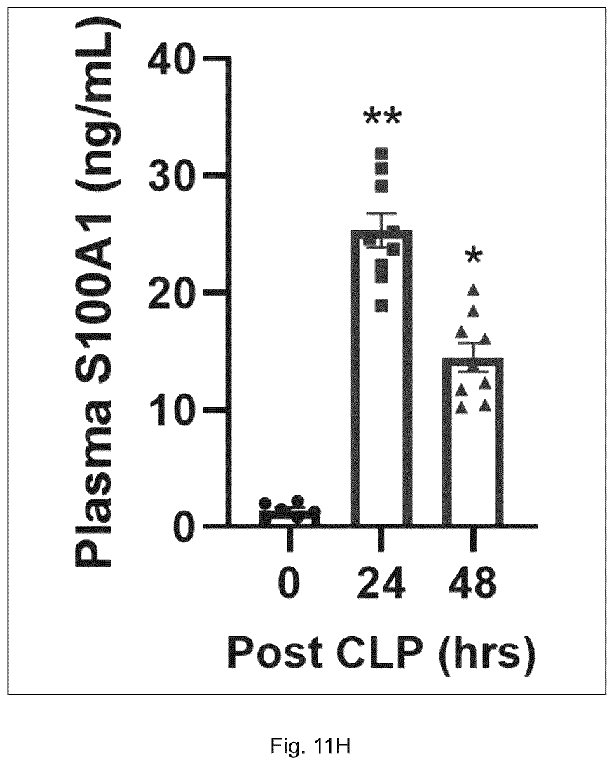

FIGS. 11A through 11I show that S100A1 is a target of miR-187-3p and that S100A1 deficient mice are resistant to sepsis. FIG. 11A illustrates that primary murine neonatal cardiomyocytes were transfected with the full-length S100A1 3′UTR fused to the luciferase (LUX) expression vector and treated with either miR-187 scrambled (SCR), miR-187-3p mimic (MIM) or inhibitor (INH); alone or with LPS (1 μg/mL) for 24 hrs (FIG. 11A and FIG. 11B). FIG. 11C shows Western blot (N=1) of S100A1 protein levels in cardiomyocytes. FIG. 11D illustrates S100A1KO-derived neonatal cardiomyocytes were treated with LPS (1 μg/mL) for 24 hrs. Bars are mean±SEM fold change (FC) in gene/18S, *p<0.05 vs. control, *p≤0.05 vs. LPS (n=3-8). Neonatal cardiomyocytes were transfected with an S100A1 expression or an empty vector and 6 hrs later treated with LPS (1 μg/mL) for 24 hrs. FIG. 11E shows mean±SEM relative to vehicle/empty vector showing increased expression of TNFα compared to LPS, *p<0.05 vs. vehicle. #p≤0.05 vs. LPS (n=3). S100A1KO and WT mice (10-12 weeks) were randomized to CLP or sham surgery. FIG. 11F shows probability of survival; *p<0.001 vs. WT-CLP. FIG. 11G provides mean±SEM % ejection fraction (EF) measured at 48 hrs post-CLP. *p<0.05 vs. WT-Sham, n=5-10. FIG. 11H provides plasma levels of S100A1 (ng/ml) measured at 24 and 48 hrs. Bars are mean±SEM, *p<0.05, **p<0.01 vs. pre-CLP, n=5-10. FIG. 11I shows Western blot (N=1) of changes in 100A1 protein levels in hearts from CLP-mice treated with LNP-miR-187-3p.

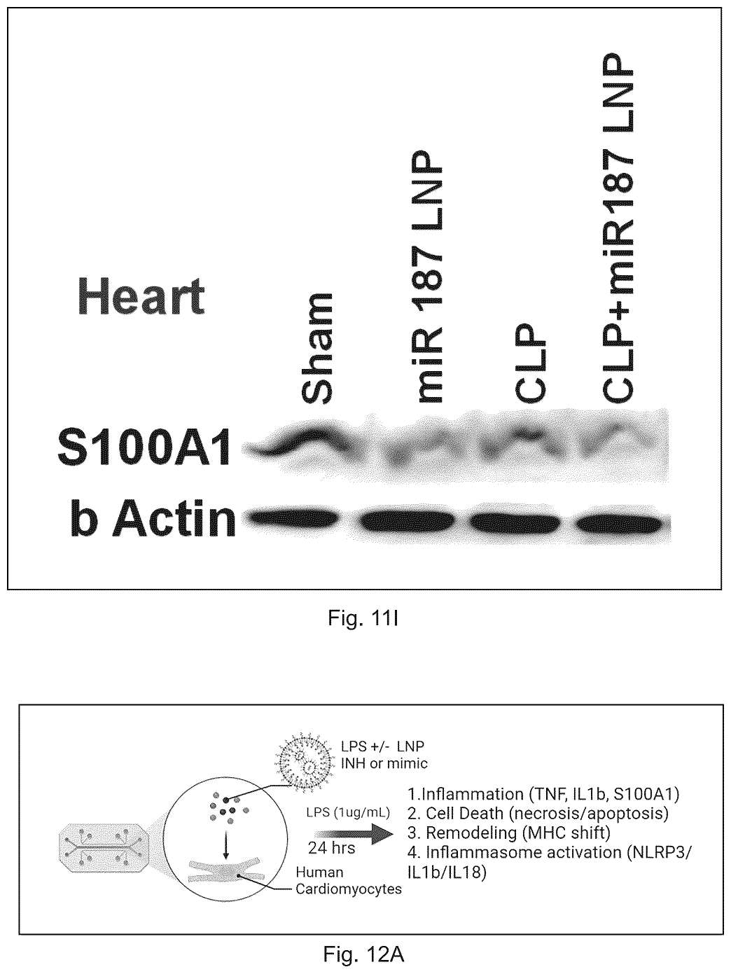

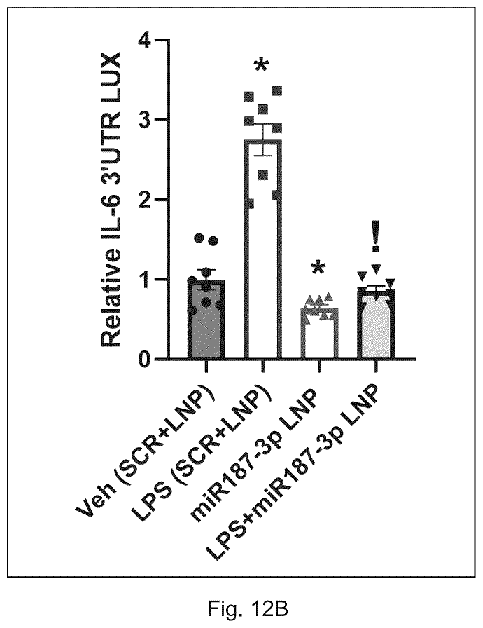

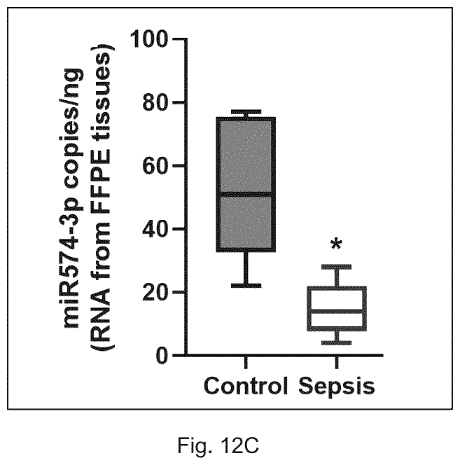

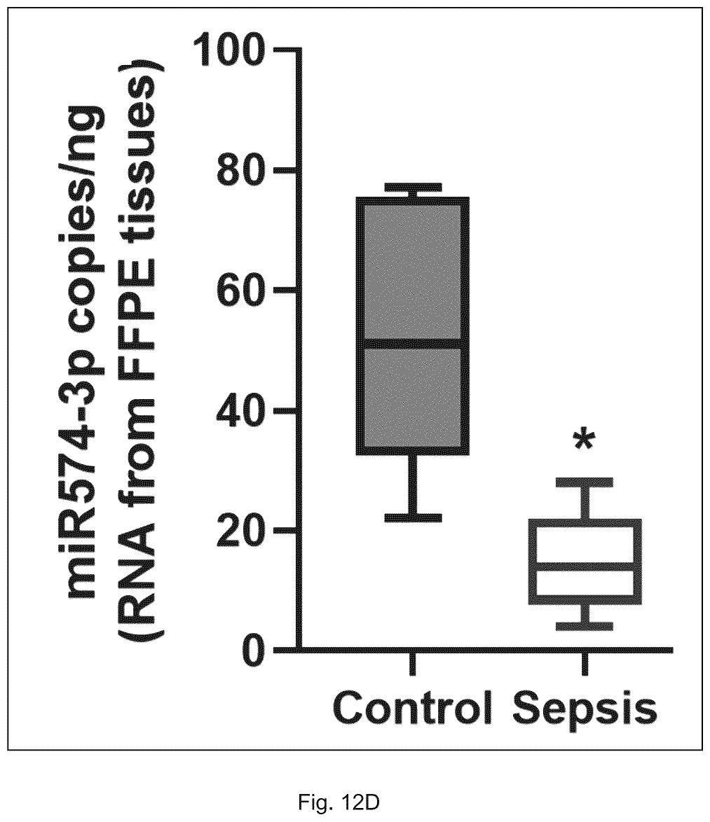

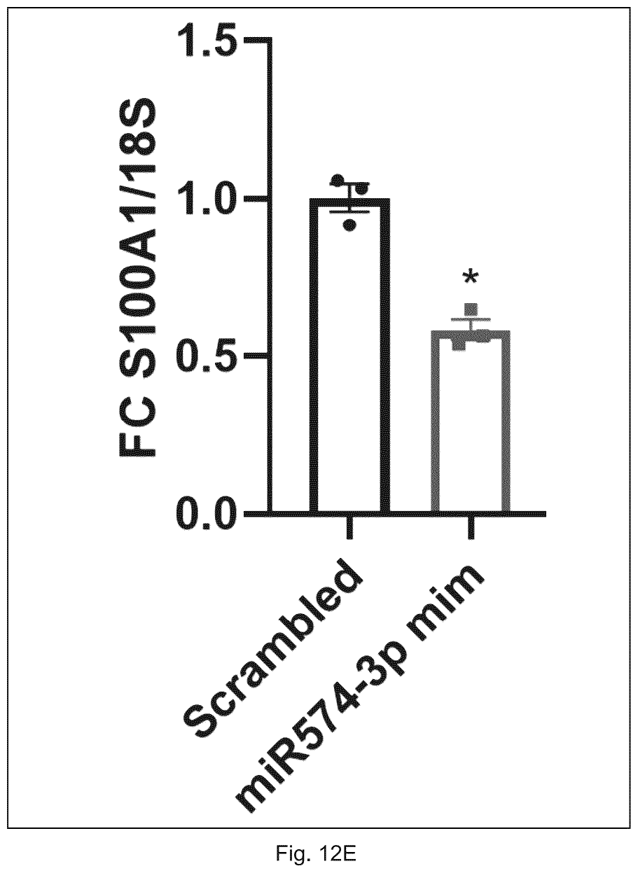

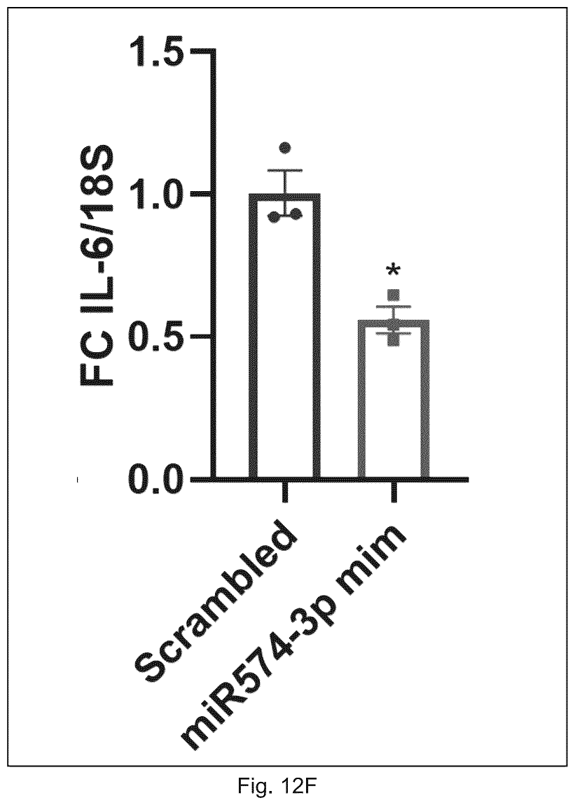

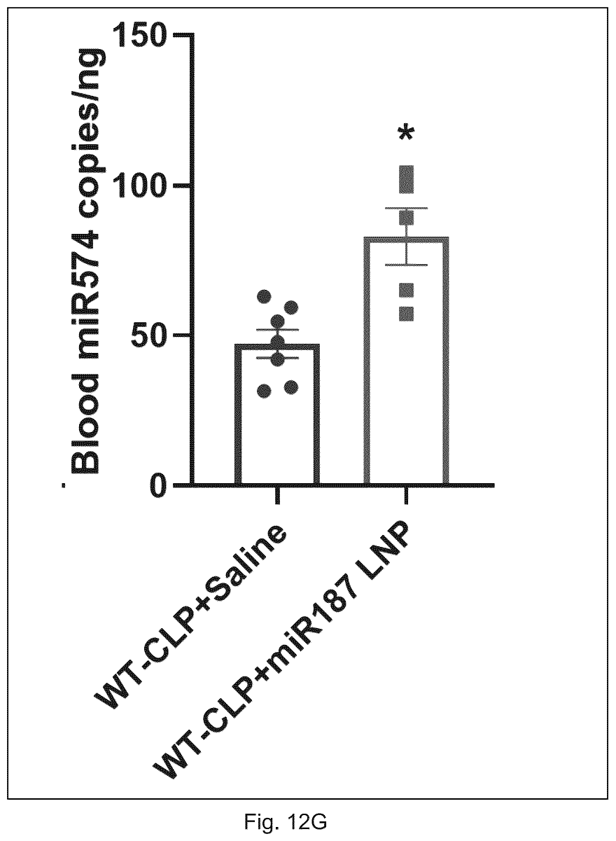

FIGS. 12A-12g show novel miRs and targets: FIG. 12A provides a schematic to test effects of the top 10 miRs identified in ci-EVs in human cardiomyocytes. FIG. 12B shows where neonatal cardiomyocytes were transfected with an IL-6 3′UTR LUX expression plasmid or empty plasmid and treated with LPS (10 μg/mL for 24 h). Bars are mean+SEM of relative LUX activity (*p<0.05 vs. vehicle; *p<0.05 vs. LPS, n=8). This direct targeting of the IL-6 3′-UTR is a novel function of miR-187-3p. FIG. 2C provides a box plot of miR-574-3p/U6 from formalin fixed paraffin embedded post-mortem human hearts from patients who died with sepsis (N=6) vs. non-septic (N=6). Neonatal cardiomyocytes were treated with LPS (IO μg/mL) or miR574-3p mimic (IO nM) for 24 hrs. Bars are mean±SEM of miR574-3p/U6 (FIG. 12D), S IO0AI/18S (FIG. 12E), and IL-6/18S (FIG. 12F); *p<0.05 vs. vehicle/scrambled, N=3. FIG. 12G provides mean±SEM of miR574-3p (copies/ng) in WT mice 48 hrs post CLP treated with saline or miRI87 LNP, *p<0.05 vs. saline, (N=5-7).

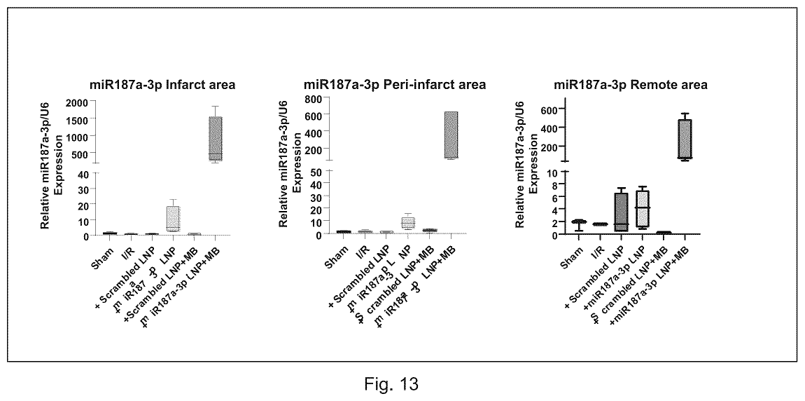

FIG. 13 shows ultrasound targeted microbubble destruction (UTMD)-mediated delivery of miR187a-3p enhanced miR187a-3p expression in the myocardium. Fisher rats (8-10 weeks old) underwent miR187a-3p-mimic LNP or scrambled LNP delivery alone or in combination with microbubbles (MB) via post-ischemia/reperfusion (I/R) or sham surgery. Data presented as box plots showing median and interquartile range relative to sham of miR187a-3p/U6 expression in the infarct (left), peri-infarct (middle) and remote (right) myocardium. *p<0.05 vs. sham. n=2-6. Note data are not normally distributed hence expressed as box plots. The LNPs were mixed with 1×10⁹ microbubbles in a 3 mL saline solution and injected to the rat via the jugular vein. For ultrasound-targeted microbubble destruction (UTMD), high-power ultrasound at a pulsing interval of 10 cardiac cycles at end-systole was transmitted to the left ventricle via a transducer at a frequency of 5 MHz, 2 cm depth, power 120 V during intravenous infusion of LNP-cationic microbubble complexes via the jugular vein over 5 minutes. The probe was positioned to cut the LV in a transverse plane (short axis) at the midpapillary level, and the transducer was slowly moved from the base to the apex to allow maximal myocardial delivery. Ultrasound transmission was continued for an additional 25 minutes after LNP-microbubble infusion to ensure maximal delivery.

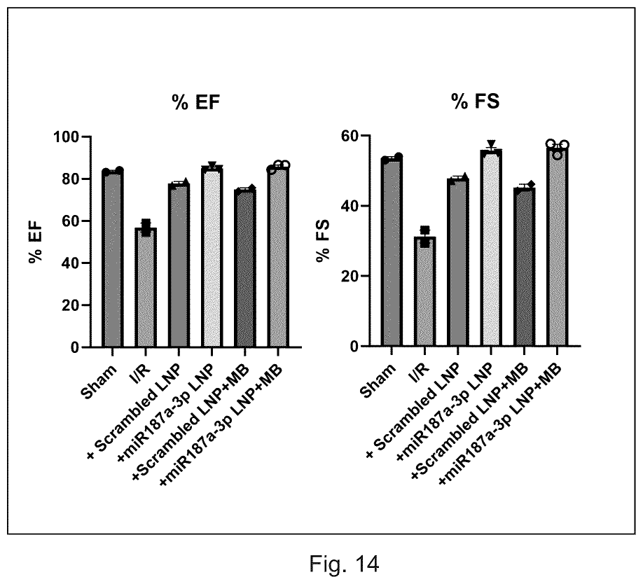

FIG. 14 shows post-I/R delivery of miR187a-3p improves echo-derived parameters of LV function. Bars are mean+SEM of echo-derived ejection fraction (left), fractional shortening (right) 3 days post-I/R-treatment with miR187a-3p LNP, scrambled LNP alone or in combination with microbubbles (MB) or sham surgery. *p<0.05 vs. sham. n=2-3/group. Very surprisingly, the dosing necessary to achieve the observed effect was about 100 times lower than necessary if the therapeutic was delivered systemically without a local triggered release using ultrasound. This greatly improves the therapeutic index.

The inventors further discovered that miR-193b-5p also binds to the 3′UTR of surfactant protein C (SPC). This direct targeting was not experimentally known previously. This is relevant because SPC deficiency occurs in acute respiratory distress syndrome (ARDS).⁵¹,⁵² While surfactant replacement therapy is used in the management of pediatric ARDS, this has not been successful in adults.⁵³ Genetic replacement of SPC attenuates lung injury and increased pathogen clearance in ALI.⁵⁴ Inhibition of miR-193b-5p may circumvent the problem of SPC replacement by preventing SPC degradation—adding value to the MoA of miR-193b-5p inhibition as a desired therapy for ALI/ARDS

In our novel discovery, to demonstrate SPC and SPD are specific targets of miR-193b-5p, the 3′UTR of each gene was cloned into a luciferase reporter construct. Decreased luciferase activity was seen in constructs containing a point mutation for the miR-193b-5p binding site in the 3′UTR of each surfactant gene.

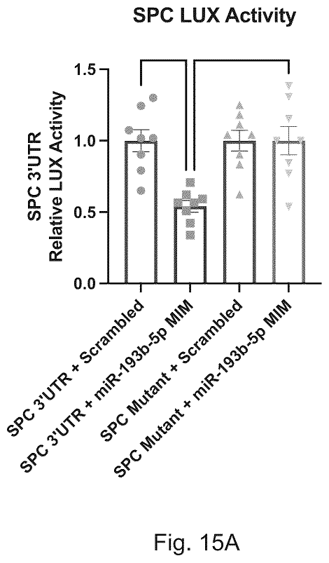

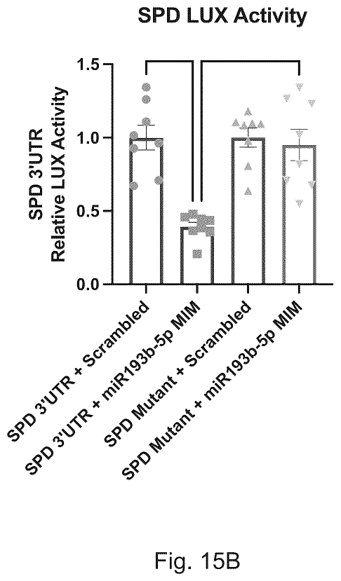

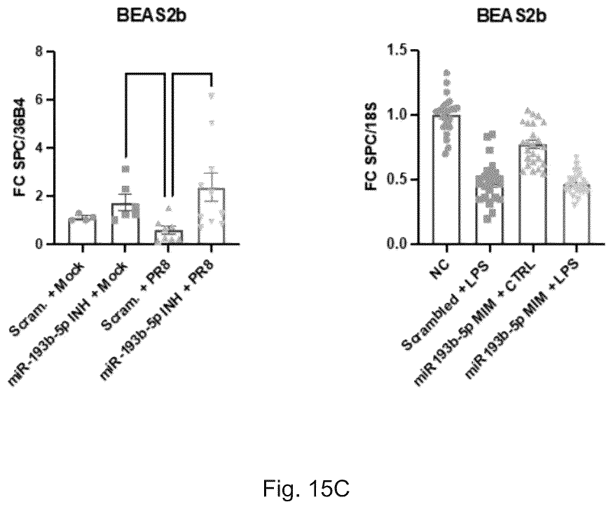

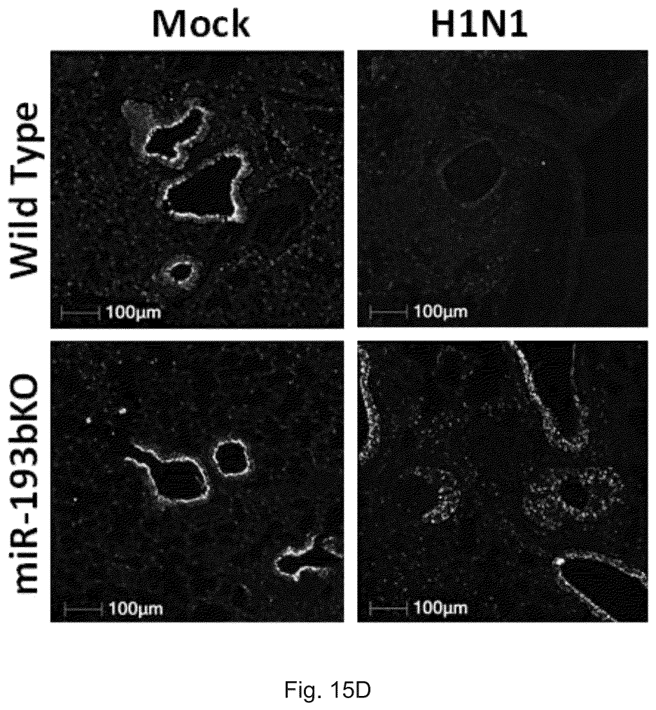

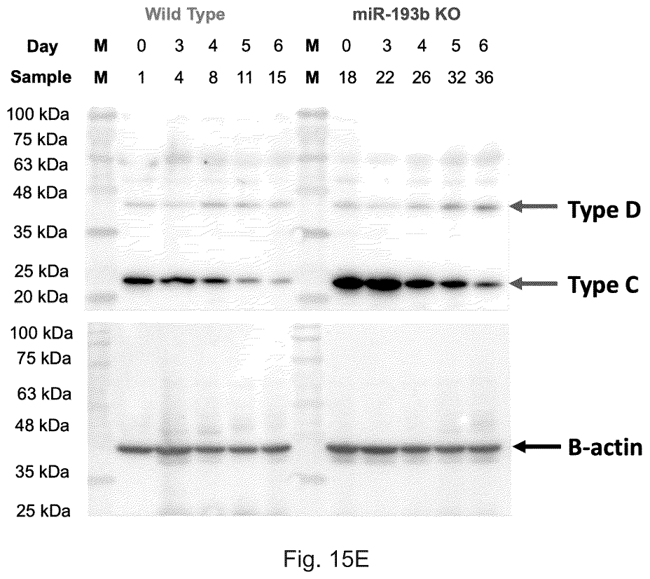

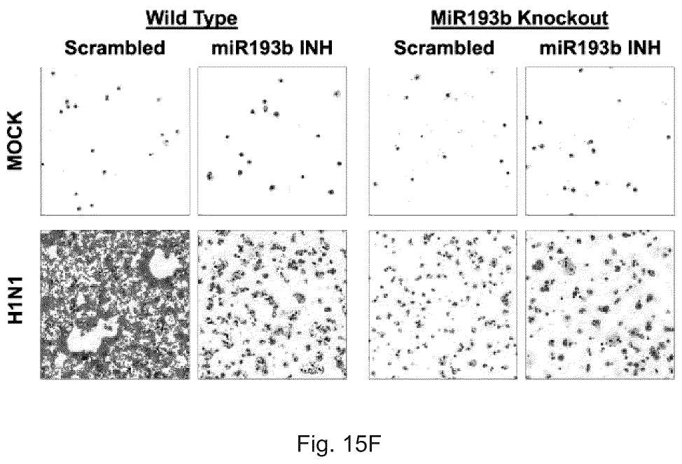

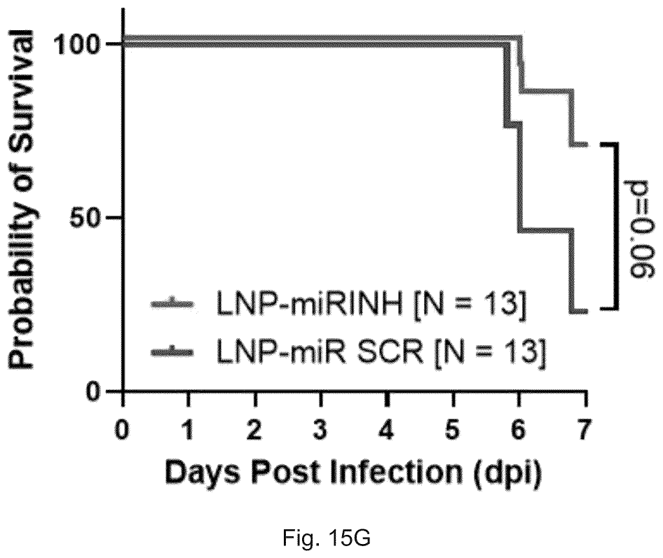

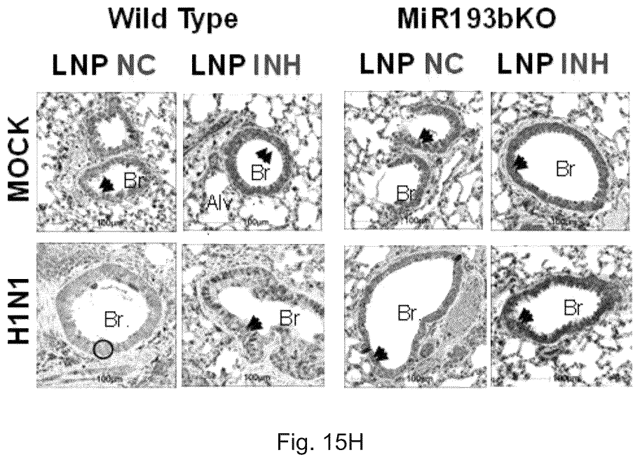

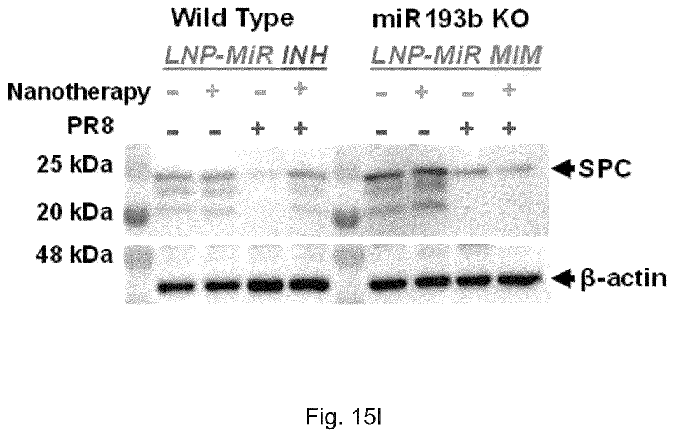

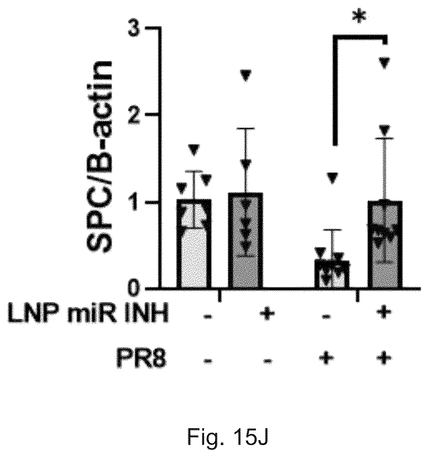

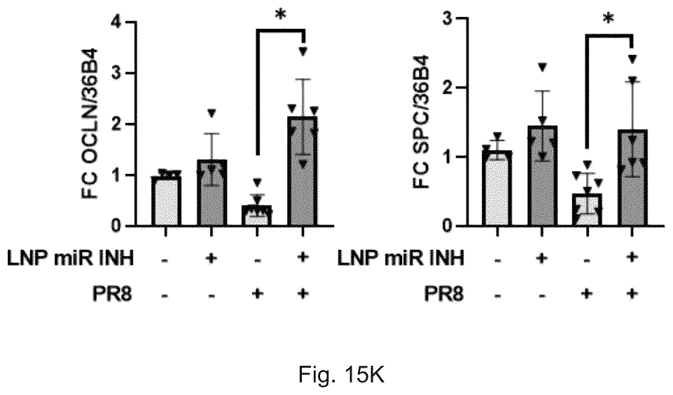

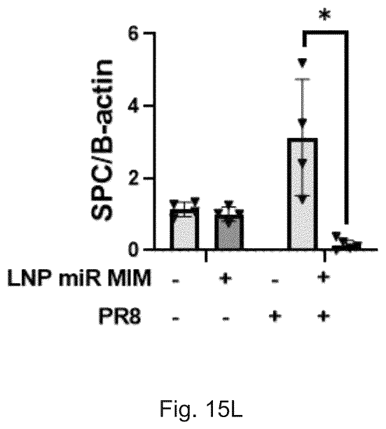

FIG. 15A shows LNP-miR-193b-5p mimic downregulates Surfactant Protein C 3′UTR luciferase activity. BEAS2b cells were co-transfected with the miR193b-5p mimic (MIM) or its scrambled (SCR) control and a Luciferase (LUX) reporter construct where we cloned either the 3′UTR wild type (WT) or mutated miR-193b-5p seed sequence (point mutation) for SPC. Bars are mean±SD LUX activity normalized to SCR, *p<0.05 vs SCR, n=5-8.

FIG. 15B shows LNP-miR-193b-5p mimic downregulates surfactant protein D 3′UTR luciferase activity. BEAS2b cells were co-transfected with the miR193b-5p mimic (MIM) or its scrambled (SCR) control and a Luciferase (LUX) reporter construct where we cloned either the 3′UTR wild type (WT) or mutated miR-193b-5p seed sequence (point mutation) for SPD. Bars are mean±SD LUX activity normalized to SCR, *p<0.05 vs SCR, n=5-8.

FIG. 15C shows the effect of miR-193b-5p modulations on surfactant protein C (SPC) expression in BEAS2b cells exposed to viral (PR8) and bacterial (LPS) models. The panel of the left shows the results of quantitative RT-PCR and shows SPC mRNA levels in PR8-infected cells treated with the LNP-miR-193b-5p inhibitor or scrambled control, while the panel on the right shows the effect of LPS exposure. Data are means±SD, n=3-6; statistical significance indicated (*p<0.05, **p<0.01, ***p<0.001, ****p<0.0001).

Wild type (WT) or miR-193b knockout mice (miR-193bKO) litter mates (8-12 weeks) were intranasally (IN) infected with H1N1 (PR8 strain A/Puerto Rico/8/1934). Infection with a median tissue culture infective dose [TCID] of 107 results in 90-100% mortality by day 7-8; mice develop progressive weight loss, hypothermia, and arterial hypoxemia.

FIG. 15D shows immunofluorescence demonstrating SPC protein (yellow) preservation in representative whole lung sections from miR-193b knockout (KO) mice relative to wild-type (WT) 6 days post-PR8/H1N1 infection. WT or miR-193bKO mice were randomized to mock (equal volume saline) or PR8/HIN1 infection.

FIG. 15E shows a representative Western Blot (WB) showing an attenuated decrease in SPC (Type C arrow) and SPD (Type D arrow) protein expression in miR-193b-5pKO compared to wild type over the 6 days post PR8/H1N1 infection.

FIG. 15F shows representative images of cytospin slides from the Bronchoalveolar lavage fluid (BALf) showing LNP-miR-193b-5p INH mitigates total cell infiltration in infected WT mice treated with LNP-miR193b-5p INH and protection in the miR-193b KO with or without treatment. Mice were randomized to mock or PR8/HIN1 infection treated at 4 days post-infection (dpi) with intravenous LNP-miR-193b-5pINH or LNP-miR-SCR-INH. Effects on lung injury and gene targets were assessed at 48 hours post-LNP delivery and the probability of survival at 7 days post-infection. R (NC is LNP-miR-SCR-INH).

FIG. 15G shows WT mice have improved survival to 7 days post PR8/H1N1 infection when treated with the LNP-miR-193bp-5pINH versus the LNP-miR-SCR-INH. WT mice were randomized to mock or PR8/HIN1 infection and treated intravenously 4 days post-infection (dpi) with LNP-miR-193b-5pINH or LNP-miR-SCR-INH. The image shows the probability of the survival log-rank test, which was p=0.06 (n=13 per group).

FIG. 15H shows WT or miR-193bKO were randomized to mock or PR8/HIN1 infection treated at 4 days post-infection (dpi) intravenously with LNP-miR-193b-5pINH or LNP-miR-SCR-INH. Representative immunohistochemistry shows preservation of OCln in mice that received LNP-miR-193b-5pINH.

FIG. 15I shows representative Western blot showing improved lung SPC protein levels from PR8/H1N1 infected mice treated with LNP-miR-193b-5p INH or LNP-193b-5p mimic (MIM) in the WT and miR-193b KO mice.

FIG. 15J shows representative Western blot demonstrating relative preservation of lung SPC protein levels in infected mice treated with LNP-miR-193b-5p-INH compared to LNP-miR-193b-5p MIM. Bars are mean±SEM.

FIG. 15K shows Western blot densitometry showing preservation of lung SPC protein level expression from infected mice treated with LNP-193b-5p-INH. Bars are mean+SEM.

FIG. 15L shows Western blot densitometry showing loss of lung SPC protein level expression from infected mice treated with LNP-193b-5p-MIM. Bars are mean+SEM.

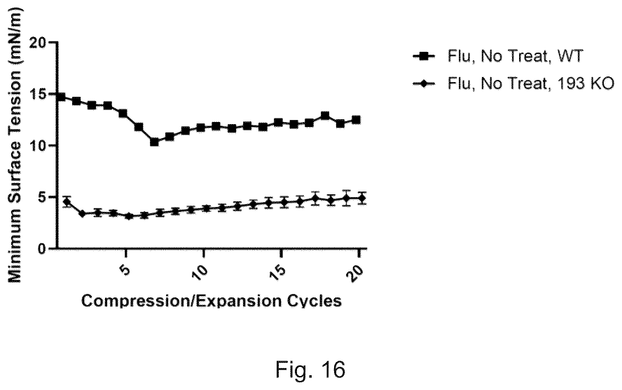

FIG. 16 shows flu-infected miR-193b KO mice show preservation of surface tension over 20 compression and expansion cycles to assess the surfactant biophysical functional activity from the Bronchoalveolar lavage fluid (BALf) collected 5 days post-PR8/H1N1 infection. This assay was performed using a constrained sessile drop surfactometer (CDS), a device used to measure pulmonary surfactants' biophysical properties.

The experiment on an miR-193b-5p knock out mouse further supports the inventors' discovery. Referring to FIG. 16, the flu-infected wild-type mouse shows the increased bronchial alveolar lavage (BALF) surface tension as high (reduced quantity of lung surfactant protein C due to the increases of miR-193b-5p), as opposed to the KO mouse, which is unable to produce miR-193b-5p.

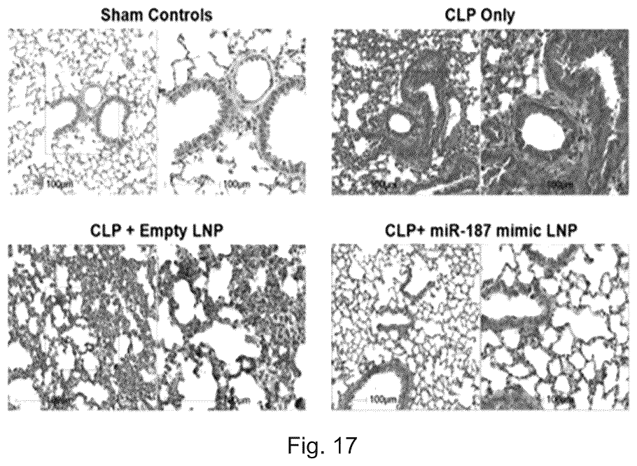

FIG. 17 shows lung tissue histology illustrating the beneficial effect of LNP-miR-187-3p after cecal ligation and puncture (CLP) two days after injection. Referring to FIG. 17, lung tissue histology illustrates the beneficial effects of miR-187-3p after cecal ligation and puncture (CLP) 2 days after injection. This discovery indicates that miR-187-3p plays additional therapeutical roles in organs other than the heart, namely the lungs.

In additional to the primary organ target of influenza viruses in mammals, the lungs, the brain is also affected. We examined the possibility that miR193b-5p plays a role in blood brain barrier (BBB) dysfunction in influenza and also that its inhibition could protect or prevent the BBB damage and astrocytes activation.

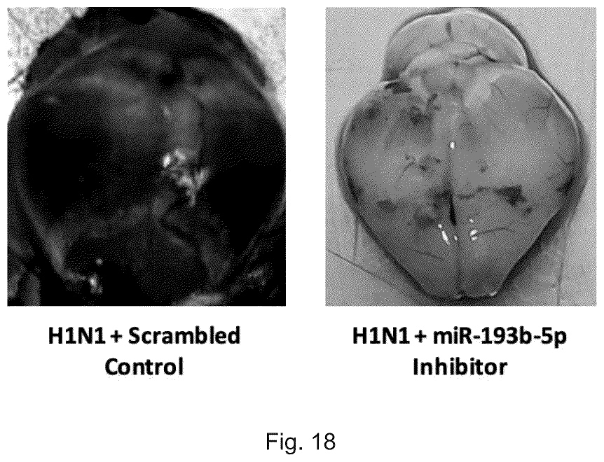

FIG. 18 shows representative gross images of mouse brains with an evident decrease in hemorrhage in miR-193b-5p inhibitor treatment through systemic injection relative to scrambled control 6 days post infection.

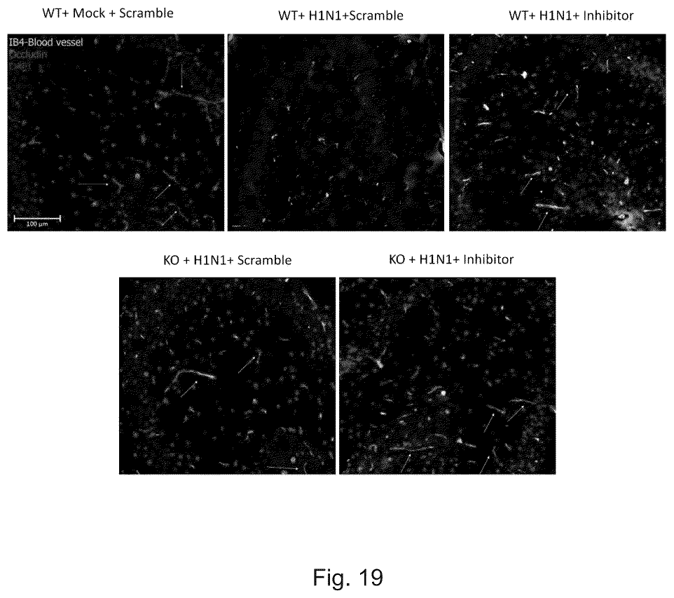

FIG. 19 shows occludin expression in the blood vessels of the hippocampus from animals infected with influenza and treated with LNP-miR193-5p-INH. Brain coronal sections show the hippocampus of WT vs miR193b KO mice counterstained with DAPI, Isolectin B-4 (IB4-blood-vessels) and immunolabeled for occludin. Scale bars: 100 μm.

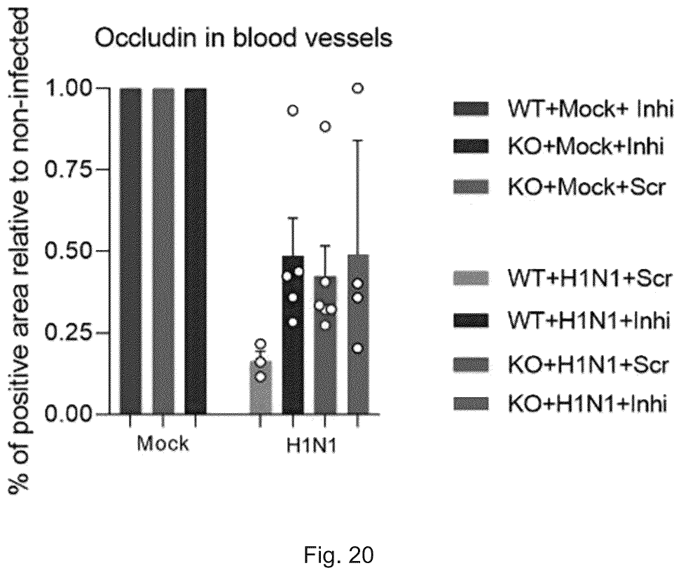

FIG. 20 shows improved preservation of occludin protein expression in the blood vessels in the hippocampus from PR8/H1N1 infected mice treated with LNP-miR193-5p-INH. Quantitative and comparative analysis of % positive stained both for occludin+IB4 and normalized to the non-infected animals brain tissues.

The BBB disruption in relate to neuroinflammatory condition, so our next step was evaluated astrogliosis—a hallmark in neuroinflammation. In addition, in infectious diseases the BBB protection could be associated with a reduction in neuroinflammation. Thus, in order to assess astrogliosis we measured the GFAP expression in the hippocampus of the animals infected with H1N1 and treated or not with miR193 inhibitor. WT animals infected with H1N1 and treated with miR193 scramble, presented an increase in GFAP expression 4× more when, when compared with non-infected animals. The treatment with miR193 inhibitor decreases the GFAP levels leading to values similar to those ones expressed by non-infected animals. This indicates a reduction in reactive astrogliosis.

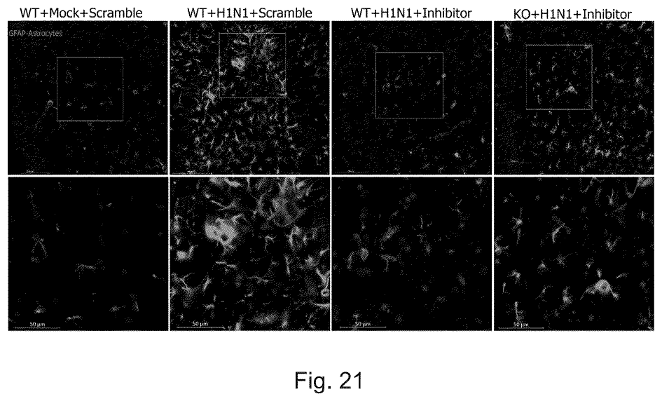

FIG. 21 shows astrocytes from mouse hippocampal regions using brain coronal sections show decreased reactivity to PR8/H1N1 infected mice treated with LNP-miR193-5p-INH and miR-193b KO mice, relative to scrambled or WT control, respectively. Brain sections were immunolabeled for GFAP and stained with DAPI. The lower row of images provides an expanded view from within the box in the image above it. Scale bars: 100 μm and 50 μm.

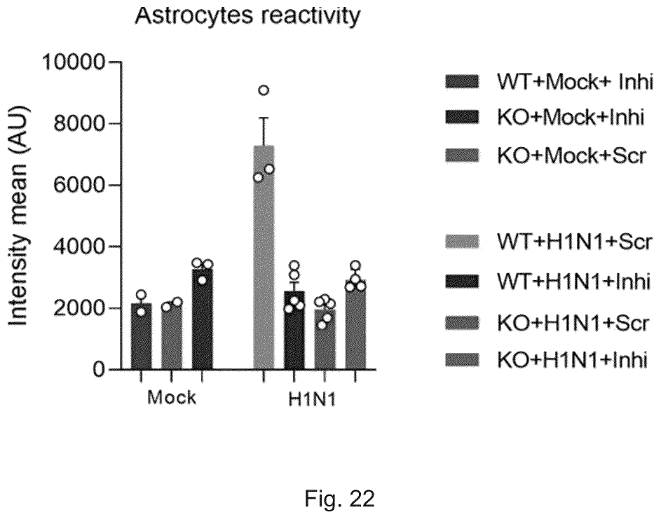

FIG. 22 shows quantitative analysis using the mean intensity values (AU) to measure astrocyte reactivity in the hippocampus of infected animals treated with the LNP-miR193-5p-INH or LNP-miR-INH-SCR (n=3 to 5 per group).

Astrocytes are one of the main cellular components of the BBB-in association with endothelial cells, pericytes, basal membrane and tight junctions they form the barrier structure. The direct association between astrocytes and blood vessels is a good indicator of the BBB integrity.

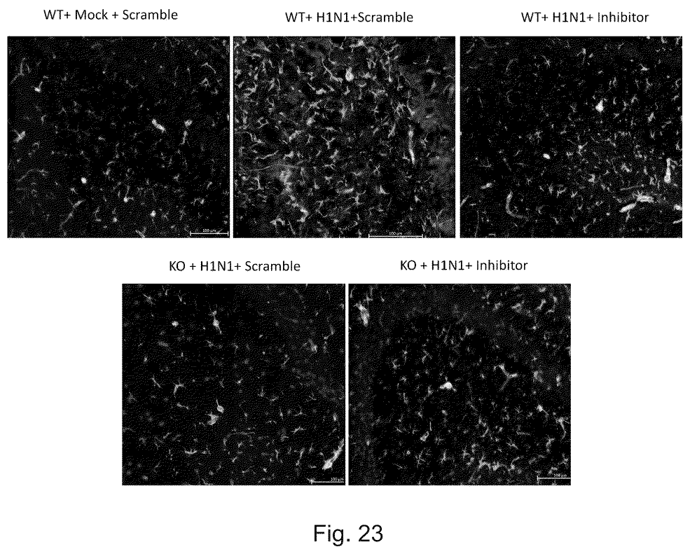

FIG. 23 shows brain coronal sections showing astrocytes more intimately associated with blood vessels in the hippocampal brain region of infected animals treated with the LNP-miR193-5p-INH relative to the LNP-miR-INH-SCR. WT (top row) and KO (bottom row) mice were counterstained with DAPI, Isolectin B-4 (IB4-blood-vessels), and immunolabeled for GFAP. Scale bars: 100 μm.

### Significance and Impact

Our innovation predicts new approaches for RNAbased therapeutics, to help elucidate the disconnect between mortality and inflammation, and unlock unknown disease processes. Our approach can be used to yield a library of LNP/RNAs that, may be used alone or in combination, to advance the care of the septic patient through personalization (determining miR levels in humans and using replacement or inhibitory therapy as appropriate) and precision (targeting specific treatable traits⁵⁵). The strength of the innovation lies in the potential for fast-tracking translation of innovative therapeutics to the clinic.

### REFERENCES

### Patent Citations

1. WO2020219941A1 2019 Apr. 26 2020 Oct. 29 Genevant Sciences Gmbh Lipid nanoparticles.
2. U.S. Pat. No. 11,141,378B2 2022 Sep. 20 2030 Jun. 30 Arbutus Biopharma Lipid formulations for delivery of therapeutic agents.
3. CN105555279B 2019 Dec. 6 2034 Jun. 25 London Health Sciences Centre Research Inc MicroRNA inhibition for treatment of sepsis.

### Non-Patent Citations

¹Butt Y., Kurdowska A., Allen T. C. Acute Lung Injury: A Clinical and Molecular Review. Arch Pathol Lab Med. 140, 345-350 (2016).
²Sweeney R. M., Griffiths M., McAuley D. Treatment of acute lung injury: current and emerging pharmacological therapies. Semin Respir Crit Care Med. 34, 487-498 (2013).
³Bruscella, P. et al. Viruses and miRNAs: More Friends than Foes. Front Microbiol 8, 824 (2017).
⁴Dos Santos, C. C. et al. Mesenchymal stromal (stem) cell therapy modulates miR-193b-5p expression to attenuate sepsis-induced acute lung injury. Eur Respir J 59, 2004216 (2022).
⁵Lee, L. K., Medzikovic, L., Eghbali, M., Eltzschig, H. K. & Yuan, X. The Role of MiRs in Acute Respiratory Distress Syndrome and Sepsis, From Targets to Therapies: A Narrative Review. Anesth Analg 131, 1471-1484 (2020).
⁶Younes, N. et al. Mesenchymal stromal/stem cells modulate response to experimental sepsis-induced lung injury via regulation of miR-27a-5p in recipient mice. Thorax 75, 556-567 (2020).
⁷Casey, J. D. et al. Use of pragmatic and explanatory trial designs in acute care research: lessons from COVID-19. Lancet Respir Med 10, 700-714 (2022).
⁸L'Heureux, M., Sternberg, M., Brath, L., Turlington, J. & Kashiouris, M. G. Sepsis-Induced Cardiomyopathy: a Comprehensive Review. Curr Cardiol Rep 22, 35 (2020).
⁹Bréchot, N. et al. Venoarterial extracorporeal membrane oxygenation to rescue sepsis-induced cardiogenic shock: a retrospective, multicentre, international cohort study. Lancet 396, 545-552 (2020).
¹⁰Jeong, H. S., Lee, T. H., Bang, C. H., Kim, J.-H. & Hong, S. J. Risk factors and outcomes of sepsis-induced myocardial dysfunction and stress-induced cardiomyopathy in sepsis or septic shock: A comparative retrospective study. Medicine (Baltimore) 97, e0263 (2018).
¹¹Liu, Y.-C., Yu, M.-M., Shou, S.-T. & Chai, Y.-F. Sepsis-Induced Cardiomyopathy: Mechanisms and Treatments. Front Immunol 8, 1021 (2017).
¹²Hollenberg, S. M. & Singer, M. Pathophysiology of sepsis-induced cardiomyopathy. Nat Rev Cardiol 18, 424-434 (2021).
¹³Martin, L. et al. The Septic Heart: Current Understanding of Molecular Mechanisms and Clinical Implications. Chest 155, 427-437 (2019).
¹⁴Shao, M. et al. miR-21-3p regulates AGE/RAGE signalling and improves diabetic atherosclerosis. Cell Biochem Funct 38, 965-975 (2020).
¹⁵Wei, J.-L. et al. LncRNA NEAT1 promotes the progression of sepsis-induced myocardial cell injury by sponging miR-144-3p. Eur Rev Med Pharmacol Sci 24, 851-861 (2020).
¹⁶Cao, C. et al. Attenuation of Sepsis-Induced Cardiomyopathy by Regulation of MicroRNA-23b Is Mediated Through Targeting of MyD88-Mediated NF-κB Activation. Inflammation 42, 973-986 (2019).
¹⁷Lima, J. F., et al. Anti-miRNA oligonucleotides: A comprehensive guide for design, RNA Biology, 15, 338-352 (2018).
¹⁸Kulkarni, J. A., Witzigmann, D., Thomson, S. B. et al. The current landscape of nucleic acid therapeutics. Nat. Nanotechnol. 16, 630-643 (2021).
¹⁹Tenchov, R. et al, Lipid Nanoparticles—From Liposomes to mRNA Vaccine Delivery, a Landscape of Research Diversity and Advancement, ACS Nano 15, 16982-17015 (2021).
²⁰Chen S, et al Dexamethasone prodrugs as potent suppressors of the immunostimulatory effects of lipid nanoparticle formulations of nucleic acids. J Control Release. 286, 46-54 (2018).
²¹Wang J, et al. MiR-101a loaded extracellular nanovesicles as bioactive carriers for cardiac repair. Nanomedicine. 27, 102201 (2020).
²²Ogawa, K. et al. Focused ultrasound/microbubbles-assisted BBB opening enhances LNP-mediated mRNA delivery to brain, J Control Release, 348, 34-41 (2022).
²³Patel, S. et al. Naturally-occurring cholesterol analogues in lipid nanoparticles induce polymorphic shape and enhance intracellular delivery of mRNA Nature communications 11, 1-13 (2020).
²⁴Cheng Q., Wei T., Farbiak L., Johnson L. T., Dilliard S. A., Siegwart D. J. Selective organ targeting (SORT) nanoparticles for tissue-specific mRNA delivery and CRISPR-Cas gene editing. Nat Nanotechnol. 15, 313-320 (2020).
²⁵Dos Santos, C. C. et al. Mesenchymal stromal (stem) cell therapy modulates miR-193b-5p expression to attenuate sepsis-induced acute lung injury. Eur Respir J 59, 2004216 (2022).
²⁶Vaswani C M, Varkouhi A K, Gupta S, Ektesabi A M, Tsoporis J N, Yousef S, Plant P J, da Silva A L, Cen Y, Tseng Y C, Batah S S, Fabro A T, Advani S L, Advani A, Leong-Poi H, Marshall J C, Garcia C C, Rocco P R M, Albaiceta G M, Sebastian-Bolz S, Watts T H, Moraes T J, Capelozzi V L, Dos Santos C C. Preventing occludin tight-junction disruption via inhibition of microRNA-193b-5p attenuates viral load and influenza-induced lung injury. Mol Ther. 2023 Sep. 6; 31(9):2681-2701.
²⁷Younes, N. et al. Mesenchymal stromal/stem cells modulate response to experimental sepsis-induced lung injury via regulation of miR-27a-5p in recipient mice. Thorax 75, 556-567 (2020).
²⁸Guttman, J. A. & Finlay, B. B. Tight junctions as targets of infectious agents. Biochimica et Biophysica Acta (BBA)—Biomembranes 1788, 832-841 (2009).
²⁹Kim, Y.-K. RNA therapy: rich history, various applications and unlimited future prospects. Exp Mol Med 54, 455-465 (2022).
³⁰Terada, T. et al. Characterization of Lipid Nanoparticles Containing Ionizable Cationic Lipids Using Design-of-Experiments Approach. Langmuir 37, 1120-1128 (2021).
³¹Kulkarni, J. A. et al. On the Formation and Morphology of Lipid Nanoparticles Containing lonizable Cationic Lipids and siRNA. ACS Nano 12, 4787-4795 (2018).
³²Matthay, M. A., Zemans, R. L., Zimmerman, G. A. et al. Acute respiratory distress syndrome. Nat Rev Dis Primers 5, 18 (2019).
³³Borger, V. et al. International Society for Extracellular Vesicles and International Society for Cell and Gene Therapy statement on extracellular vesicles from mesenchymal stromal cells and other cells: considerations for potential therapeutic agents to suppress coronavirus disease-19. Cytotherapy 22, 482-485 (2020).
³⁴Witwer, K. W. et al. Defining mesenchymal stromal cell (MSC)-derived small extracellular vesicles for therapeutic applications. J Extracell Vesicles 8, 1609206 (2019).
³⁵Börger, V., Staubach, S., Dittrich, R., Stambouli, O. & Giebel, B. Scaled Isolation of Mesenchymal Stem/Stromal Cell-Derived Extracellular Vesicles. Curr Protoc Stem Cell Biol 55, e128 (2020).
³⁶Madel, R. J. et al. Independent human mesenchymal stromal cell-derived extracellular vesicle preparations differentially affect symptoms in an advanced murine Graft-versus-Host-Disease model. 2020.12.21.423658 Preprint at https://doi.org/10.1101/2020.12.21.423658 (2020).
³⁷Théry, C. et al. Minimal information for studies of extracellular vesicles 2018 (MISEV2018): a position statement of the International Society for Extracellular Vesicles and update of the MISEV2014 guidelines. Journal of Extracellular Vesicles 7, 1535750 (2018).
³⁸Welsh, J. A. et al. A compendium of single extracellular vesicle flow cytometry. J Extracell Vesicles 12, e12299 (2023).
³⁹Van Hoecke, L. et al. Anti-Inflammatory Mesenchymal Stromal Cell-Derived Extracellular Vesicles Improve Pathology in Niemann-Pick Type C Disease. Biomedicines 9, 1864 (2021).
⁴⁰Tertel, T., Görgens, A. & Giebel, B. Analysis of individual extracellular vesicles by imaging flow cytometry. Methods Enzymol 645, 55-78 (2020).
⁴¹Varkouhi, A. K. et al. Immunophenotypic characterization and therapeutics effects of human bone marrow- and umbilical cord-derived mesenchymal stromal cells in an experimental model of sepsis. Experimental Cell Research 399, 112473 (2021).
⁴²Mei, S. H. et al. Mesenchymal stem cells reduce inflammation while enhancing bacterial clearance and improving survival in sepsis. American journal of respiratory and critical care medicine 182, 1047-1057 (2010).
⁴³Lalu, M. M. et al. Evaluating mesenchymal stem cell therapy for sepsis with preclinical metaanalyses prior to initiating a first-in-human trial. eLife 5, e17850 (2016).
⁴⁴Matthay, M. A. Extracellular Vesicle Transfer from Mesenchymal Stromal Cells Modulates Macrophage Function in Acute Lung Injury. Basic Science and Clinical Implications. Am J Respir Crit Care Med 196, 1234-1236 (2017).
⁴⁵Silva, J. D. et al. Mesenchymal Stromal Cells Are More Effective Than Their Extracellular Vesicles at Reducing Lung Injury Regardless of Acute Respiratory Distress Syndrome Etiology. Stem Cells Int 2019, 8262849 (2019).
⁴⁶Sarker, S. et al. Placenta-derived exosomes continuously increase in maternal circulation over the first trimester of pregnancy. J Transl Med 12, 204 (2014).
⁴⁷Valadi, H. et al. Exosome-mediated transfer of mRNAs and microRNAs is a novel mechanism of genetic exchange between cells. Nat Cell Biol 9, 654-659 (2007).
⁴⁸Mendelson, A. A. et al. National Preclinical Sepsis Platform: developing a framework for accelerating innovation in Canadian sepsis research. Intensive Care Medicine Experimental 9, 14 (2021).
⁴⁹Home. Sepsis Canada https://www.sepsiscanada.ca/?resourceID=1.
⁵⁰Labusek et al. Inflammation and Regeneration (2023) 43:24
⁵¹Veldhuizen, R. A., McCaig, L. A., Akino, T. & Lewis, J. F. Pulmonary surfactant subfractions in patients with the acute respiratory distress syndrome. Am J Respir Crit Care Med 152, 1867-1871 (1995).
⁵²Han, S. & Mallampalli, R. K. The Role of Surfactant in Lung Disease and Host Defense against Pulmonary Infections. Ann Am Thorac Soc 12, 765-774 (2015).
⁵³Ruaro, B. et al. Severe COVID-19 ARDS Treated by Bronchoalveolar Lavage with Diluted Exogenous Pulmonary Surfactant as Salvage Therapy: In Pursuit of the Holy Grail? J Clin Med 11, 3577 (2022).
⁵⁴Kesecioglu, J. Farewell to exogenous surfactant therapy in acute lung injury/acute respiratory distress syndrome! Or, must we start all over again? Crit Care Med 38, 1606-1607 (2010).
⁵⁵Lee Y Q, Selvakumar A, See K C. Treatable Traits in Chronic Respiratory Disease: A Comprehensive Review. Cells. 2021 Nov. 22; 10(11):3263.

1. A method for treating or preventing sepsis in a subject in need, comprising steps of:
administering miRNA-based particles to a subject in need, and
observing extended survival of a subject suffering from sepsis and reduced organ dysfunction or failure caused by the condition;
wherein each of said miRNA-based particles comprises miR-193b-5p inhibitor, encapsulated in a lipid nanoparticle carrier.

2. The method according to claim 1, wherein the step of administering the miRNA-based particles includes injecting a physiologically acceptable solution containing the miRNA-based particles into the subject.

3. The method according to claim 1, wherein the step of administering the miRNA-based particles includes mixing the miRNA-based particles with microbubbles to adhere a plurality of the miRNA-based particles to outer surfaces of the microbubbles and injecting the microbubbles into a patient, followed by applying ultrasound at one or more targeted locations of the patient's body to rupture the microbubbles causing release of the miRNA-based particles, and/or the cargo of the miRNA-based particles.

4. The method according to claim 1, wherein the method is for treating dysregulated immune response induced by the sepsis.

5. The method according to claim 1, wherein the method is for treating acute respiratory distress syndrome induced by the sepsis.

6. The method according to claim 1, wherein the method is for preventing or treating neuroinflammation induced by the sepsis.

7. The method according to claim 1, wherein the method is for preventing lung injury.

8. The method according to claim 1, wherein the miRNA-based particles further comprise miR-187-3p mimic encapsulated in a lipid nanoparticle carrier.

9. The method according to claim 8, wherein the method is for any one or any combination of:
treating acute respiratory distress syndrome,
preventing or treating neuroinflammation,
preventing lung injury, and
treating or preventing myocardial dysfunction.

10. The method according to claim 1, wherein said miR-193b-5p inhibitor is an miRNA sponge comprising synthetic or naturally occurring RNA molecules, said miRNA sponge having multiple binding sites for miR-193b-5p.

11. The method according to claim 1, wherein the miR-193b-5p inhibitor is a synthetic single-stranded oligonucleotide having 18-24 monomers in length that bind to miRNA-binding region of target genes, wherein the miR-193b-5p inhibitor optionally further includes a longer guide strand integrated with one or more passenger strands.

12. The method according to claim 11, wherein the miRNA-binding region is 3′-untranslated region of surfactant protein C gene.

13. The method according to claim 11, wherein the miRNA-binding region is 3′-untranslated region of occludin gene.

14. The method according to claim 11, wherein a backbone of the oligonucleotide includes one or more phosphorothioate (PS) that replace the natural phosphodiester (PO) bond in naturally occurring oligonucleotides.

15. The method according to claim 11, wherein one or more ribose sugars of the oligonucleotide include modifications at the 2′-O positions, said modifications being 2′-O-methyl (2′-OMe), 2′-O-methoxyethyl (2′MOE), or 2′-fluoro (2′-F).

16. The method according to claim 11, wherein the oligonucleotide comprises conformationally constrained analogues to RNA.

17. The method according to claim 16, wherein, the conformationally constrained analogues to RNA includes constrained 2′-O-ethyl (cEt), locked nucleic acid (LNA), or 2′-O,4′-C-ethylene-bridged nucleic acids (ENA) prepared by putting in a methyl bridge from 2′-O to 4′-C positions of the ribose sugar, that replace one or more of the naturally occurring RNA bases.

18. The method according to claim 1, wherein the miRNA-based particles further comprise a pro-drug.

19. The method according to claim 1, wherein the lipid nanoparticle carrier comprises at least four lipids, comprising: a) an ionizable cationic lipid positively charged in a formulation buffer, b) a sterol, c) a structural helper lipid, and d) a PEGylated-lipid, said at least four lipids being independent from each other.

20. The method according to claim 19, wherein the ionizable cationic lipid is positively charged in a formulation buffer having a pH in a range from about pH 3 to about pH 5.5.

21. The method according to claim 19, wherein the ionizable cationic lipid is neutral in a storage buffer having a pH in a range from about pH 7 to about pH 8.

22. The method according to claim 19, wherein the ionizable cationic lipid is any one or a combination of saturated lipids, unsaturated lipids, single-tail lipids, multi-tail lipids, polymeric lipids, biodegradable lipids, or branched tail lipids.

23. The method according to claim 19 wherein the ionizable cationic lipid comprises neutral or true fats, waxes, cutin, suberin, phospholipids, sphingolipids, lipoproteins, terpenes, prostaglandins, or sterols.

24. The method according to claim 19 wherein the structural helper lipid c) is a sterol that is independent from the sterol b).

25. The method according to claim 19, wherein the structural helper lipid is cholesterol.

26. The method according to claim 19, wherein the PEGylated-lipid is polyethylene glycol (PEG) derivatives attached to a lipid moiety.

27. The method according to claim 19, wherein the PEGylated-lipid is DMG-PEG2000 or 1,2-dimyristoyl-rac-glycero-3-methoxypolyethylene glycol-2000 ALC-0159 or (2-hexyldecanoate), 2-[(polyethylene glycol)-2000]-N,N-ditetradecylacetamide DSPE-PEG, DPPE-PEG, DOPE-PEG, DMPE-PEG with PEG lengths varying from 0.2 to 5 kDa.

28. The method according to claim 19, wherein a ratio of the ionizable lipid, the sterol, the structural helper lipid, and the PEGylated-lipid in a mol % ratio range of about 40-70:30-45:3-16:0.5-1.5.

29. The method according to claim 1, wherein the lipid carrier is a cell membrane derived nano-vesicle.

30. The method according to claim 29, wherein the miRNA-based particles further comprise miR-187-3p mimic.
